(19) United States
(12) Patent Application Publication
(10) Pub. No.: US 2004/0219202 A1
(43) Pub. Date: Nov. 4, 2004

(54) Pharmaceutical composition comprising lipids comprising a polar and a nonpolar moiety

(76) Inventors: Steven Fletcher, Wolsingham (GB); Michael R. Jorgensen, London (GB); Andrew David Miller, Chiswick (GB)

Correspondence Address: Scott A McCollister, Fay Sharpe Fagan Minnich & McKee, 7th Floor, 1100 Superior Avenue, Cleveland, OH, 44114-2518, US

(21) Appl. No.: 10/484,855

(22) PCT Filed: Jul. 29, 2002
(86) PCT No.: PCT/GB02/03488

(30) Foreign Application Priority Data
Jul. 30, 2001 (GB) 0118517.2

(51) Int. Cl. A61K 9/127; A61K 31/225

(52) U.S. Cl. 424/450; 514/547

(57) ABSTRACT

The present invention provides a composition comprising (i) a lipid compound comprising at least one non-polar moiety and a polar moiety, wherein the non-polar moiety is of the formula X-Y-Z- wherein X is an acetylenic hydrocarbyl group containing a single C≡C bond, Y is O or CH₂, and Z is an optional hydrocarbyl group, wherein the polar moiety is of the formula -[T]ₘPHG, wherein [T]ₘ is an optional group selected from C(O), NH, NR₁, NHC(O), C(O)NH, NRIC(O) and C(O)NR₁ and CH₂, where R₁ is a hydrocarbyl group, wherein PHG is a polar head group, and wherein m is the number of non-polar moieties (ii) a therapeutic agent.

The present invention relates to a composition. In addition, the present invention relates to a compound and to a liposome and to the use of the composition, compound or liposome in therapy, in particular gene therapy (especially gene delivery).

One aspect of gene therapy involves the introduction of foreign nucleic acid (such as DNA) into cells, so that its expressed protein may carry out a desired therapeutic function.^{1a}

Examples of this type of therapy include the insertion of TK, TSG or ILG genes to treat cancer; the insertion of the CFTR gene to treat cystic fibrosis; the insertion of NGF, TH or LDL genes to treat neurodegenerative and cardiovascular disorders; the insertion of the IL-1 antagonist gene to treat rheumatoid arthritis; the insertion of HIV antigens and the TK gene to treat AIDS and CMV infections; the insertion of antigens and cytokines to act as vaccines; and the insertion of β-globin to treat haemoglobinopathic conditions, such as thalassaemias.

Many current gene therapy studies utilise adenoviral gene vectors—such as Ad3 or Ad5- or other gene vectors. However, serious problems have been associated with their use.^{2a} This has prompted the development of less hazardous, non-viral approaches to gene transfer.^{3a}

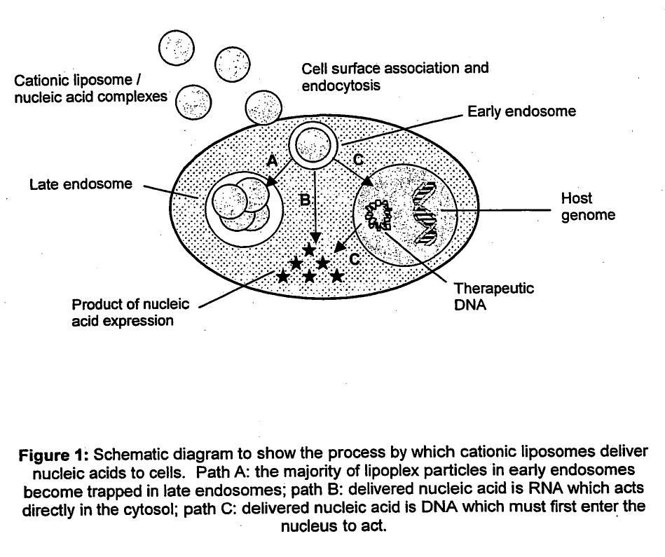

Non-viral vectors are also known in the art. In essence, non-viral gene therapy requires a vector that is capable of mimicking viruses, yet is non-pathogenic. On the basis that nucleic acid is negatively-charged, there has been developed two types of cationic non-viral vector (both of which serve to condense the nucleic acid): liposomes and polymers. Their resulting complexes with DNA (lipoplexes and polyplexes, respectively) are still cationic, thereby facilitating endocystosis (cellular uptake) at the anionic cell surface. Once internalised, the complex may then suffer three fates shown in FIG. 1.

A non-viral transfer system of great potential involves the use of cationic liposomes.^{4a} In this regard, cationic liposomes—which usually consist of a neutral phospholipid and a cationic lipid—have been used to transfer DNA^{4a}, mRNA^{5a}, antisense oligonucleotides^{6a}, proteins^{7a}, and drugs^{8a} into cells. A number of cationic liposomes are commercially available^{4a,9a} and many new cationic lipids have recently been synthesised^{10a}. The efficacy of these liposomes has been illustrated by both in vitro^{4a} and in vivo^{11a}.

Liposomes are formed by the molecular self-assembly of lipids. Cationic liposomes are often formulated with a mixture of both cationic lipids and neutral, “helper” lipids. DOPC (1) and DOPE (2) are two such neutral helper lipids, so-called because they tend to improve the transfection abilities of cationic liposomes and may also help cationic lipids to form liposomes. Moreover, 2 is the most popular of all the helper lipids and has been unequivocally proven to improve transfection (gene delivery and expression) of its constituent lipoplexes, attributed to its peculiar fusogenic properties.^{2b} Conversely, DODAP (3) (at pH 4.0)^{2c} and DOTAP (4) are both cationic lipids. These structures electrostatically bind DNA.

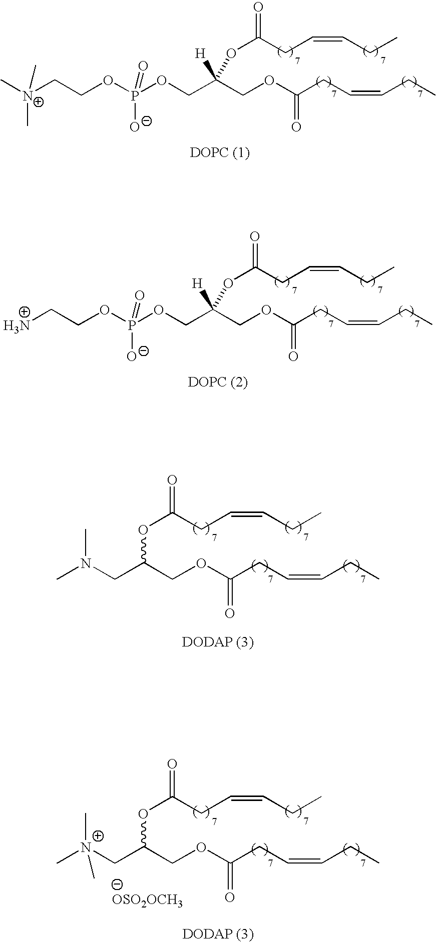

One of the most commonly used cationic liposome systems consists of a mixture of a neutral phospholipid dioleoylphosphatidylethanolamine (commonly known as “DOPE”) and a cationic lipid, 3β-[N-(N′,N′-dimethylaminoethane)carbamoyl]cholesterol (commonly known as “DC-Chol”)^{12a}.

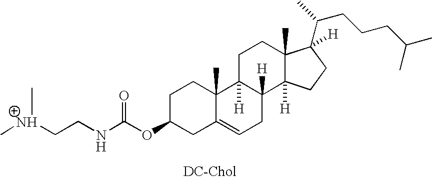

Liposomes have already proven their worth as agents for drug delivery with a number of formulations having reached clinical trials,^{6b} efficiently encapsulating the drug molecule to be delivered within their aqueous interiors. Cationic liposomes interact electrostatically with plasmid DNA, but do not, as such, encapsulate the nucleic acid. Such resulting particles, termed lipoplexes (LDs), transfect well in vitro.

Despite the efficacy of the known cationic liposomes there is still a need to optimise the gene transfer efficiency of cationic liposomes in human gene therapy^{10a}. With the near completion of the human genome project, the use of genes for therapeutic purposes, described as gene therapy is increasingly expected to revolutionise medicine. In this context, even though still less effective than viral technology, non-viral delivery is increasingly recognised by the scientific community as the safest option for human applications.

This field has evolved considerably in the last decade with the apparition of complex macromolecular constructs including many elements of different existing technologies (viral proteins or peptides, liposomes, polymers, targeting strategies and stealth properties).

WO 01/48233 teaches a system based on a triplex composed of a viral core peptide Mu, plasmid DNA and cationic Liposome (LMD). This platform technology gave us good success in vitro and promising results in vivo. But as for all existing non-viral technology more development is needed to achieve a therapeutic level in vivo.

To this end, formulation must achieve stability of the particle in biological fluids (serum, lung mucus) and still maintain efficient transfection abilities.

This requirement is one of the main hurdles of all existing technology. Current stable formulations^{[1,2]} achieve little transfection and most present efficient transfecting agents are drastically limited in the scope of their application due to this instability^{[3-7]}.

After administration (in blood for systemic application or in mucus for lung topical administration), the charged complexes are exposed to salt and biological macromolecules leading to strong colloidal aggregation and adsorption of biological active elements (opsonins) at their surface^{[8-11]}. The gene vehicles undergo drastic changes that could include precipitation, binding of proteins leading to particle elimination by macrophages and surface perturbation resulting in its destruction. The most widely used stabilised formulation involves surface-grafted polyethylene glycol (PEG) chains^{[12, 13]} PEG is a non-toxic, neutral polyether which has a large exclusion volume for most macromolecules. Unfortunately formulations demonstrating the necessary level of stabilisation are reported to loose their gene transfer ability, probably due to their reduced non-specific affinity for cells or the loss of their necessary endosome breaking properties^{[14, 15]}.

An alternative approach to escaping the destructive effect of biological fluid on lipoplexes is to attempt to mimic nature and coat the surface of lipid bilayers with polysaccharides^{[16, 17]}.

In 1991, it was reported that octadeca-9-ynoic acid (6) shows DNA binding properties.^{8b} Octadeca-9-ynoic acid has an apparent DNA dissociation constant of 1.8 mM; it inhibits topisomerase I-mediated DNA filter binding but does not inhibit DNA topoisomerase I-mediated relaxation of a supercoiled plasmid DNA. Furthermore, the fatty acid is weakly inhibitory to DNA polymerase α.

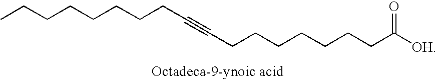

Platelet lipoxygenase (LOX) may be selectively inhibited by acetylenic fatty acids. For example, octadeca-9,12-diynoic acid irreversibly inactivates Fe(III)-LOX.^{9b}

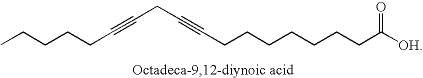

Cytochrome P4504A4 (CYP4A4) is a pulmonary cytochrome P450 which metabolises prostaglandins and arachidonic acid into their ω-hydroxylated products. Prostaglandins play important roles in the regulation of reproductive, vascular and inflammatory systems. Octadeca-17-ynoic acid has been shown to be an effective inhibitor^{10b} of the substrate-binding pocket of CYP4A4.

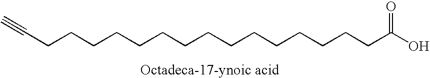

Octadeca-5-ynoic acid (tariric acid) inhibits the hatching of C. tomentosicollis eggs.^{11b}

Undeca-10-ynoic acid, an inhibitor of cytochrome P4504A1, inhibits ethanolamine-specific phospholipid base exchange reaction in rat liver microsomes.^{12b}

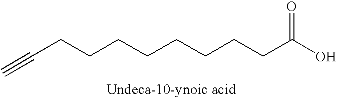

A number of acetylenic fatty acids, such as octadeca-8,10,12-triynoic acid, have shown to be potent inhibitors of the enzyme cyclo-oxygenase and weak inhibitors of 5-lipoxygenase.^{13b}

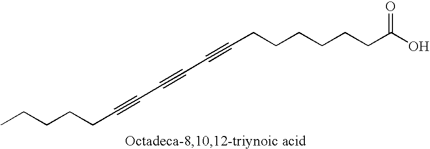

The acetylenic fatty acid eicosa-5,8,11-triynoic acid inhibits mammalian hepatic glutathione S-transferases.^{14b}

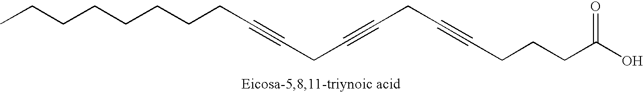

Liposomes incorporating fluorescent and/or metal-chelating lipids offer potential applications as probes in the world of cellular biology, such as monitoring the progress of encapsulated DNA in non-viral gene therapy. Conjugated, diacetylenic lipids have been prepared with ethylenediaminetetra-acetic acid (EDTA) head-groups, capable of chelating lanthamide ions.^{15b} These lipids (each with two conjugated, acetylenic fatty acyl groups (e.g. diacetylenic)) have been successfully incorporated into liposomes and then allowed to polymerise. Fluorescence studies indicate that the diacetylenic functionality (unpolymerised lipid) and the conjugated alkenes (after polymerisation) can be used as sensitizers for the lanthanide ions.

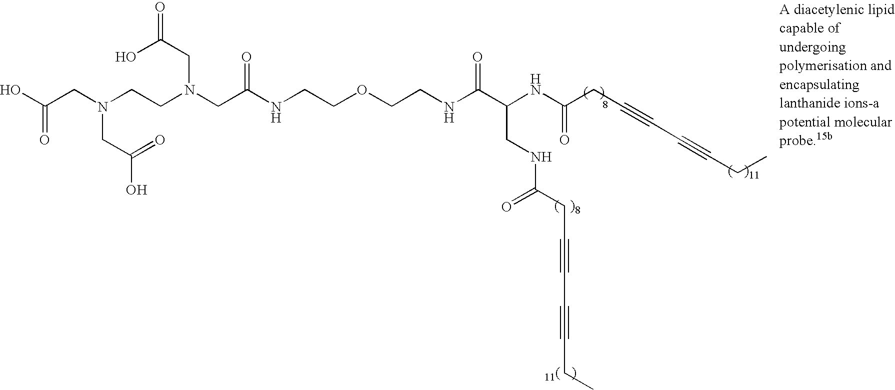

Diacetylenic phospholipids have been shown to undergo polymerisation when incorporated into liposomes and exposed to UV light. Such a system is more stable than without polymerised lipids and is appropriate for slow-release drug delivery systems.

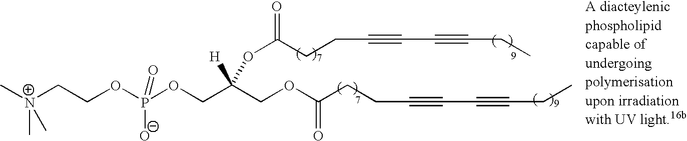

Junichi et al. ^{17b} have reported (WO 95/03035) the use of polymerised liposomes with enhanced stability for oral delivery of drugs. Pharmaceutical compounds for oral delivery can be encapsulated within polymerised liposomes, then delivered to the small intestine. The constituent phospholipids of the liposomes are polymerised through double-bond containing olefinic and acetylenic phospholipids. Such polymerisation adds strength, resulting in less fluid liposomes. For this reason, polymerisable phospholipids may offer limited use in non-viral gene therapy, unless the polymerisation process is reversible. On the one hand, the liposomes must be stable in the blood but on the other hand, they must be unstable once inside the cell; fluidity (and concomitant affinity for the H_{II} phase) appears to be all important in improving transfection in vitro.

Upon polymerisation within liposomes, diacetylenic phosphatidylcholines have shown thromboresistance in vitro. This aspect of stability may be a consequence of the polymerised phosphatidylcholines not being able to participate in coagulation.^{18b}

In 2001, it was shown that the cationic lipid bisguanidinium-diacetylene (BGDA) is highly efficient for in vitro transfection when formulated as cationic liposomes with DOPE.^{19b} The presence of the diacetylenic functionality offers the potential for polymerisation and, therefore, a novel scaffold for gene transfection. This may offer further insight into the structure-activity relationships of lipid/DNA complexes through studying the effects of polymerisable domains.

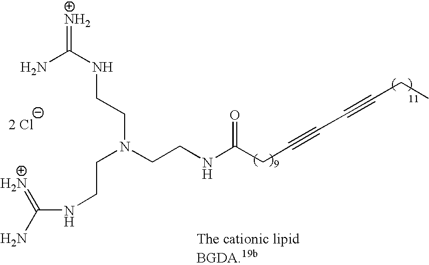

The present invention addresses the problems of the prior art.

According to one aspect of the present invention there is provided a composition comprising (i) a lipid compound comprising at least one non-polar moiety and a polar moiety, wherein the non-polar moiety is of the formula X-Y-Z-, wherein X is an acetylenic hydrocarbyl group containing a single C≡C bond, Y is O or CH₂, and Z is an optional hydrocarbyl group, wherein the polar moiety is of the formula -[T]ₘPHG, wherein [T]ₘ is an optional group selected from C(O), NH, NR₁, NHC(O), C(O)NH, NR₁C(O), C(O)NR₁ and CH₂, where R₁ is a hydrocarbyl group, wherein PHG is a polar head group, and wherein m is the number of non-polar moieties, (ii) a therapeutic agent.

According to another aspect of the present invention there is provided a liposome comprising a lipid compound, wherein the lipid compound comprises at least one non-polar moiety and a polar moiety, wherein the non-polar moiety is of the formula X-Y-Z-, wherein X is an acetylenic hydrocarbyl group containing a single C≡C bond, Y is O or CH₂, and Z is an optional hydrocarbyl group, wherein the polar moiety is of the formula-[T]ₘPHG wherein [T]ₘ is an optional group selected from C(O), NH, NR₁, NHC(O), C(O)NH, NR₁C(O), C(O)NR₁ and CH₂, where R₁ is a hydrocarbyl group, wherein PHG is a polar head group, and wherein m is the number of non-polar moieties; wherein the compound is other than DO(4-yne)PC, DO(9-yne)PC and DO(14-yne)PC.

According to another aspect of the present invention there is provided a lipid compound comprising at least one non-polar moiety and a polar moiety, wherein the non-polar moiety is of the formula X-Y-Z-, wherein X is an acetylenic hydrocarbyl group containing a single C≡C bond, Y is O or CH₂, and Z is an optional hydrocarbyl group, wherein the polar moiety is of the formula -[T]ₘPHG, wherein [T]ₘ is an optional group selected from C(O), NH, NR₁, NHC(O), C(O)NH, NR₁C(O), C(O)NR₁ and CH₂, where R₁ is a hydrocarbyl group, wherein PHG is a polar head group, and wherein m is the number of non-polar moieties;

wherein the compound is other than DO(4-yne)PC, DO(9-yne)PC, DO(14-yne)PC, DO(4-yne)PE and DO(14-yne)PE.

According to another aspect of the present invention there is provided use of a lipid compound in the manufacture of a medicament for the treatment of genetic disorder or condition or disease, wherein the compound is a lipid compound comprising at least one non-polar moiety and a polar moiety, wherein the non-polar moiety is of the formula X-Y-Z-, wherein X is an acetylenic hydrocarbyl group containing a single C≡C bond, Y is O or CH₂, and Z is an optional hydrocarbyl group, wherein the polar moiety is of the formula-[T]ₘPHG, wherein [T]ₘ is an optional group selected from C(O), NH, NR₁, NHC(O), C(O)NH, NR₁C(O), C(O)NR₁ and CH₂, where R₁ is a hydrocarbyl group, wherein-PHG is a polar head group, and wherein m is the number of non-polar moieties.

According to another aspect of the present invention there is provided a compound, composition or liposome according to the present invention for use in therapy.

According to another aspect of the present invention there is provided the use of a compound, composition or liposome in the manufacture of a medicament for the treatment of a genetic disorder or a condition or a disease.

According to another aspect of the present invention there is provided a cationic liposome formed from the compound according to the present invention or a compound when prepared by the process of the present invention.

According to another aspect of the present invention there is provided a method of preparing a cationic liposome comprising forming the cationic liposome from the compound according to the present invention or a compound when prepared by the process of the present invention.

According to another aspect of the present invention there is provided a cationic liposome according to the present invention or a cationic liposome as prepared by the method of the present invention for use in therapy.

According to another aspect of the present invention there is provided the use of a cationic liposome according to the present invention or a cationic liposome as prepared by the method of the present invention in the manufacture of a medicament for the treatment of genetic disorder or condition or disease.

According to another aspect of the present invention there is provided a combination of a nucleotide sequence and any one or more of: a compound according to the present invention, a compound when prepared by the process of the present invention, a liposome of the present invention, a liposome as prepared by the method of the present invention, a composition of the present invention, or a composition as prepared by the method of the present invention

According to another aspect of the present invention there is provided a combination according to the present invention for use in therapy.

According to another aspect of the present invention there is provided the use of a combination according to the present invention in the manufacture of a medicament for the treatment of genetic disorder or condition or disease.

According to another aspect of the present invention there is provided a pharmaceutical composition comprising a compound according to the present invention, a compound when prepared by the process of the present invention, a composition according to the present invention or a composition when prepared by the process of the present invention admixed with a pharmaceutical and, optionally, admixed with a pharmaceutically acceptable diluent, carrier or excipient.

According to another aspect of the present invention there is provided a pharmaceutical composition comprising a cationic liposome according to the present invention or a cationic liposome as prepared by the method of the present invention admixed with a pharmaceutical and, optionally, admixed with a pharmaceutically acceptable diluent, carrier or excipient.

Some further aspects of the invention are defined in the appended claims.

We have studied the issues of promoting endosomolysis (such as exploiting the fall in pH as an endosome matures into a lysosome), cell-specific targeting (lipopeptides), DNA condensation, nuclear targeting and, lastly, improving the in vivo stability, yet maintaining the fusogenicity, of the lipoplex.

It is believed that a key advantage of the compound or composition of the present invention is that it can be used in the preparation of a cationic liposome useful in gene therapy, in particular the delivery of nucleic acids (including genes and antisense DNA/RNA) into cells (in vitro, in vivo and ex vivo) to derive a therapeutic benefit.

Gene therapy agents are typically administered intravenously. Paradoxically, cationic liposomes must be stable in blood, yet unstable once inside a cell, enabling escape of delivered nucleic acid. Serum components in blood reduce biological activity of current cationic liposomes which may lead to clearance or to displacement of the therapeutic nucleic acid, hence poor in vivo results. We have found that in vitro transfection activities of acetylenic compounds of the present invention to be comparable with that of DOPE. It is believed that the carbon-carbon triple bond is more resistant to oxidation than double bonds, and may also increase membrane rigidity. The acetylenic compounds may offer greater stability in blood serum that may improved in vivo results

We have studied a broad range of compounds within the scope of the invention and have observed advantageous results. In particular, we have studied the corresponding analogues of DOPC, DOPE, DODAP and DOTAP of each of three C18 (5, 6, and 7) and two C17 (8 and 9) monoacetylenic fatty acids.

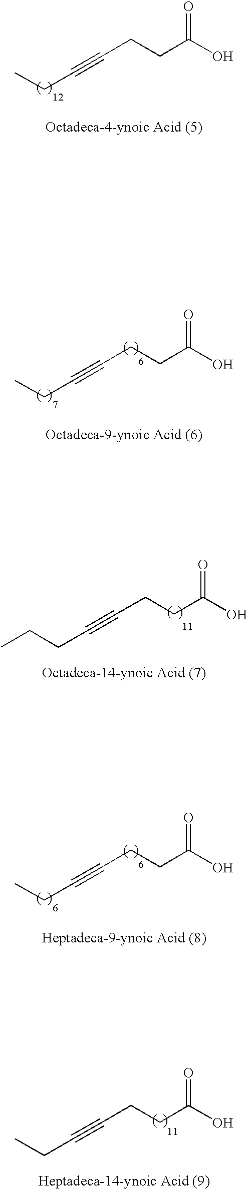

The syntheses of five monoacetylenic analogues of a number of lipids, namely DOPC (1), DOPE (2), DODAP (3) and DOTAP (4), has been completed. These lipids should be capable of better intermolecular packing, thereby affording less fluid liposome structures and enhancing the circulation lifetimes of such liposomes in vivo.

The presence of a —C≡C— triple bond is believed to be advantageous because it is less prone to oxidation than —C═C— double bond,^{20b} and, in general, less susceptible to attack by electrophilic agents.^{21b} Furthermore, the double bond is vulnerable to cis I trans isomerisation whilst no such isomerisation can occur with the triple bond. Acidic conditions and UV light can accelerate this process. It is highly probable that the cis double bonds in DOPE are essential for the transfection potency of DOPE, making DOPE-containing liposomes prone to form the H_{II} phase and, therefore, fusogenic (see below). Isomerisation to the more stable trans form may result in reduced, or even total loss of, transfection properties.^{21c}

Lipids with cis double bonds, such as DOPE, cause their constituent membranes to be fluid. Acetylenic analogues, such as DO(9-yne)PE, are believed to confer greater rigidity, therefore enhanced stability in vivo, on their constituent liposomes.

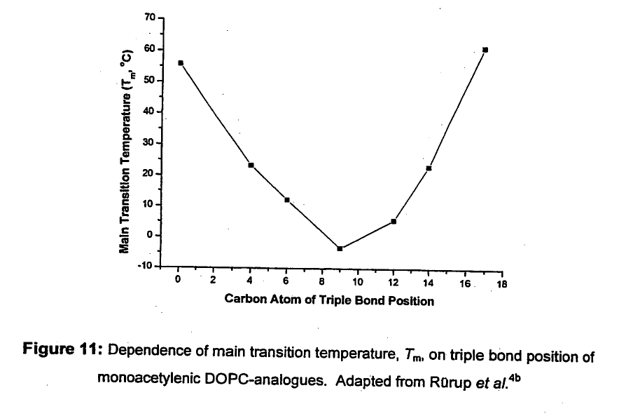

R\overline{u}rup et al.^{4b} have shown that the lamellar gel-to-liquid-crystalline (L₆₂ /L_{α}) transition temperature (Tₘ) of the 9-yne analogue of DOPC (DO(9-yne)PC, 33) occurs around 15° C. higher (−3.4° C.) than standard DOPC (−18° C.). Furthermore, they show that as the triple bond is moved in either direction away from the middle of the fatty acyl chain, the Tₘ increases (FIG. 11), as it does with the olefinic (double bond) analogues.

Accordingly, the corresponding DOPE-analogues may exhibit similar behaviour, not only for the L₆₂ /L_{α} transition but also for the L₆₀ /H_{II} phase transition. The lamellar liquid crystalline-inverted hexagonal (L_{α}/H_{II}) phase transition temperature (Tₕ) occurs at 10° C. for standard DOPE.^{21d} Due to its affinity for the H_{II}, phase, DOPE is referred to as a fusogenic lipid since, when membranes fuse, the structural intermediates are similar to those involved in bilayer (L_{α}) to H_{II} phase transitions. It is believed that it is this fusogenic property which makes DOPE-containing liposomes potent towards transfection through fusion of the liposomal and endosomal membranes, enabling the escape of the plasmid DNA into the cytoplasm.

Due to the low Tₕ, it is impossible to prepare liposomes of DOPE under the physiological conditions of 37° C. (or, indeed, at 25° C.) and pH 7.0. On the basis that DO(9-yne)PC has a Tₘ of approximately 15° C. higher than DOPC, the Tₘ of DO(9-yne)PE may be approximately 15° C. higher than DOPE. More importantly, the Th of DO(9-yne)PE may be around 15° C. higher than for DOPE. If this is the case, it may, therefore, be easier to prepare cationic liposomes containing our monoacetylenic analogues of lipids such as DOPE than it would be to use the lipid itself, at room temperature (RT). Ultimately, this could lead to lowering the positive charges of cationic LMDs, generating transfecting particles which are not only more stable due to better intermolecular packing hence more rigid bilayer structures but more stable due to the particles being more neutral. Non-viral gene therapy is hampered by a number of issues in vivo, one such problem being the fact that there are a number of negatively charged components in the blood. These may label the cationic LMDs for destruction through electrostatic interactions, or more simply may displace the anionic mu-DNA complex. The present acetylenic analogues may create more vectors which are more stable towards aggregation and towards destructive, anionic entities.

Broad Aspects of the Invnetion

The term “acetylenic hydrocarbyl” as used herein means a group comprising at least C and H, having at least one —C≡C— bond and may optionally comprise one or more other suitable substituents. Examples of such substituents may include halo-, alkoxy-, nitro-, a hydrocarbon group, an N-acyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

It will be understood by one skilled in the art that by “X is an acetylenic hydrocarbyl group containing a single C≡C bond” it is meant the or each X contains one and only one C≡C bond.

The term “hydrocarbyl group” as used herein means a group comprising at least C and H and may optionally comprise one or more other suitable substituents. Examples of such substituents may include halo-, alkoxy-, nitro-, a hydrocarbon group, an N-acyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

In one preferred embodiment of the present invention, the hydrocarbyl group is a hydrocarbon group.

Here the term “hydrocarbon” means any one of an alkyl group, an alkenyl group, an alkynyl group, an acyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In one broad aspect the present invention provides a composition comprising (i) a lipid compound comprising at least one non-polar moiety and a polar moiety, wherein the non-polar moiety is of the formula X-Y-Z-, wherein X is an acetylenic hydrocarbyl group, Y is O or CH₂, and Z is an optional hydrocarbyl group, wherein the polar moiety is of the formula -[T]ₘPHG, wherein Mm is an optional group selected from C(O), NH, NR₁, NHC(O), C(O)NH, NR₁C(O), C(O)NR₁ and CH₂, where R₁ is a hydrocarbyl group, wherein PHG is a polar head group, and wherein m is the number of non-polar moieties, (ii) a therapeutic agent.

In one broad aspect the present invention provides a liposome comprising a lipid compound, wherein the lipid compound comprises at least one non-polar moiety and a polar moiety, wherein the non-polar moiety is of the formula X-Y-Z-, wherein X is an acetylenic hydrocarbyl group, Y is O or CH₂, and Z is an optional hydrocarbyl group, wherein the polar moiety is of the formula -[T]ₘPHG, wherein [T]ₘ is an optional group selected from C(O), NH, NR₁, NHC(O), C(O)NH, NR₁C(O), C(O)NR₁ and CH₂, where R¹ is a hydrocarbyl:group, wherein PHG is a polar head group, and wherein m is the number of non-polar moieties; wherein the compound is other than DO(4-yne)PC, DO(9-yne)PC and DO(14-yne)PC.

In one broad aspect the present invention provides a lipid compound comprising at least one non-polar moiety and a polar moiety, wherein the non-polar moiety is of the formula X-Y-Z-, wherein X is an acetylenic hydrocarbyl group, Y is O or CH₂, and Z is an optional hydrocarbyl group, wherein the polar moiety is of the formula -[T]ₘPHG, wherein [T]ₘ is an optional group selected from C(O), NH, NR₁, NHC(O), C(O)NH, NR₁C(O), C(O)NR₁ and CH₂, where R₁ is a hydrocarbyl group, wherein PHG is a polar head group, and wherein m is the number of non-polar moieties; wherein the compound is other than DO(4-yne)PC, DO(9-yne)PC, DO(14-yne)PC, DO(4-yne)PE and DO(14-yne)PE.

In one broad aspect the present invention provides use of a lipid compound in the manufacture of a medicament for the treatment of genetic disorder or condition or disease, wherein the compound is a lipid compound comprising at least one non-polar moiety and a polar moiety, wherein the non-polar moiety is of the formula X-Y-Z-, wherein X is an acetylenic hydrocarbyl group, Y is O or CH₂, and Z is an optional hydrocarbyl group, wherein the polar moiety is of the formula -[T]ₘPHG, wherein [T]ₘ is an optional group selected from C(O), NH, NR₁, NHC(O), C(O)NH, NR₁C(O), C(O)NR₁ and CH₂, where R₁ is a hydrocarbyl group, wherein PHG is a polar head group, and wherein m is the number of non-polar moieties.

In some preferred embodiments of the above broad aspects of the present invention the or at least one X is an acetylenic hydrocarbyl group containing a single C≡C bond.

In one preferred embodiment of the present invention, the acetylenic hydrocarbyl group is an alkynyl group.

For ease of reference, these and further aspects of the present invention are now discussed under appropriate section headings. However, the teachings under each section are not necessarily limited to each particular section.

Preferable Aspects

The compound may be an anionic lipid.

Preferably the compound is a neutral lipid.

Preferably the compound is a cationic lipid.

Polar Moiety

Polar Head Group (PHG)

It will be appreciated by one of skill in the art that the polar head group may be derived from a suitable lipid. By the term “lipid” it may be meant a compound based on a fatty acids or a closely related compounds such as their corresponding alcohol or sphingosine base.

In one preferred aspect the polar head group is derived from phospholipids, ceramides, triacylglycerols, lysophospholipids, phosphatidylserines, glycerols, alcohols, alkoxy compounds, monoacylglycerols, gangliosides, sphingomyelins, cerebrosides, phosphatidylcholines, phosphatidylethanolamines, phosphatidylinositols (PI), diacylglycerols, Phosphatidic acids, glycerocarbohydrates, polyalcohols and phosphatidylglycerols.

In one preferred aspect the polar head group is derived from phospholipids, ceramides, triacylglycerols, lysophospholipids and phosphatidylserines.

Preferably the polar head group is derived from of a phospholipid.

Preferably the phospholipid is a neutral or anionic phospholipid.

In one preferred aspect the phospholipid is selected from phosphatidylcholine (PC) and phosphatidylethanolamine (PE), such as such as dioleoyl-L-α-phosphatidylethanolamine (DOPE).

Preferably the polar head group is derived from 3-N,N-dimethylaminopropan-1,2-diol (DAP) or 3-N,N,N-trimethylammoniopropan-1,2-diol (TAP).

In one aspect the polar head group (PHG) may be the group —W-Linker-HG, wherein W is selected from CH₂, O, NR¹ and S, wherein R¹ is H or a hydrocarbyl group, wherein Linker is an optional linker group, and HG is a head group.

The head group (HG) may be polar or non-polar. When HG is non-polar it may be rendered polar by group —C(O)W-Linker. Such head groups are encompassed by the present definition provided —C(O)W-Linker-HG is polar and HG is polar when attached to the —C(O)W-Linker-group.

In one aspect the head group (HG) may be an alkyl group. In this aspect preferably the alkyl contains at least 5 carbon, for example it is a C₅₋₁₀₀ alkyl group, a C₅₋₈₀ alkyl group, a C₅₋₆₀ alkyl group, a C₅₋₅₀ alkyl group, a C₅₋₄₀ alkyl group, a C₅₋₃₀ alkyl group or a C₅₋₂₀ alkyl group.

In one aspect the head group (HG) is derived from phospholipids, ceramides, triacylglycerols, lysophospholipids, phosphatidylserines, glycerols, alcohols, alkoxy compounds, monoacylglycerols, gangliosides, sphingomyelins, cerebrosides, phosphatidylcholines, phosphatidylethanolamines, phosphatidylinositols (PI), diacylglycerols, Phosphatidic acids, glycerocarbohydrates, polyalcohols and phosphatidylglycerols.

In one preferred aspect the head group is of the formula

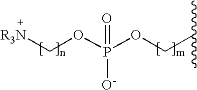

or of the formula

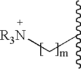

wherein R is independently selected from H and hydrocarbyl, m is from 1 to 10 and n is from 1 to 10.

Preferably R is selected from H and C₁₋₆ alkyl, more preferably from H and C₁₋₃ alkyl, more preferably from H and methyl.

Preferably m is from 1 to 5, more preferably 1, 2 or 3.

Preferably n is from 1 to 5, more preferably 1, 2 or 3.

Linker

The linker of —W-Linker-HG may be any suitable group. A typical linker group is a hydrocarbyl group.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In one preferred aspect at least one optional linker group is not present. In one preferred aspect no optional linker groups are present.

When one or more or all optional linker groups are not present, the group/compound from which the polar head group is derived is typically chosen to have one or more —OH groups. These allow a simple ester bond between the non-polar moiety and the polar moiety to be provided.

It will be appreciated by one skilled in the art that when an optional linker is present two or more W groups may or may not be bonded to the same atom of the linker. It is envisages that in some aspects the two or more W groups are boned to different atoms of a linker.

W

W of —W-Linker-HG is selected from CH₂, O, NR¹ and S, wherein R¹is H or a hydrocarbyl group.

In one preferred aspect W is O or NR¹.

R¹ is preferably H or a hydrocarbon group.

R¹ is preferably H, C₁₋₃₀, C₁₋₂₅, C₁₋₂₀, C₁₋₁₅, C¹⁻¹⁰, C₁₋₅, or C₅₋₁₅ hydrocarbyl group.

R¹ is preferably H, C₁₋₃₀, C₁₋₂₅, C₁₋₂₀, C₁₋₁₅, C₁₋₁₀, C₁₋₅, or C₅₋₁₅ hydrocarbon group.

R¹ is preferably H, C₁₋₃₀, C_{1-25 , C}₁₋₂₀, C₁₋₁₅, C₁₋₁₀ C₁₋₅, or C₅₋₁₅ optionally substituted alkyl group.

R¹ is preferably H, C₁₋₃₀, C ₁₋₂₅, ₁₋₂₀, C₁₋₁₅, C₁₋₁₀, C₁₋₅, or C₅₋₁₅ unsubstituted alkyl group.

Non-Polar Moiety

X

As discussed above X is a hydrocarbyl chain. By “hydrocarbyl chain” it is meant a linear hydrocarbyl group.

In the following definitions of chain length it is meant the longest chain of directly bonded atoms within moiety X. It will be understood that a chain does not include atoms of cyclic substituents or substituents of a terminal carbon.

In one preferred aspect X is a group selected from optionally substituted alkyl, optionally substituted alkenyl and optionally substituted alkynyl.

In one preferred aspect the acetylenic hydrocarbyl group contains from 3 to 30 carbon atoms, such as from 10 to 25 carbon atoms, from 15 to 20 carbon atoms, 15, 16, 17 or 18 carbon atoms.

Preferably the acetylenic hydrocarbyl group is derived from a fatty acid selected from

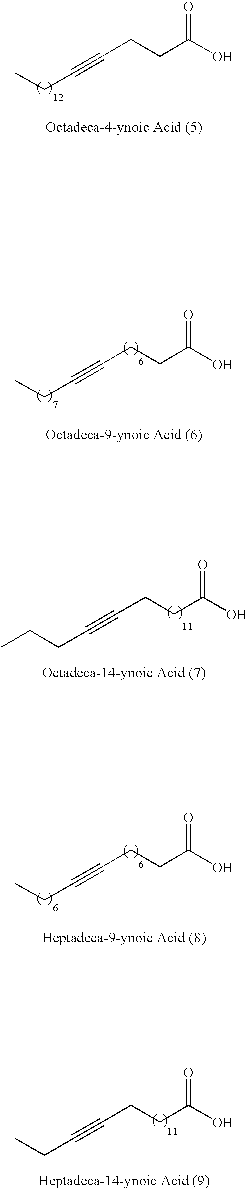

In one preferred aspect X is a group selected from optionally substituted C₆-C₂₄ alkynyl groups.

In one preferred aspect X is a group selected from optionally substituted alkynyl groups having a chain length of 6 to 24 atoms.

In one preferred aspect X is a group selected from optionally substituted: alkynyl groups having a chain length of 10 to 18 atoms.

In one preferred aspect X is a group selected from optionally substituted alkynyl groups having a chain length of 16 or 17 atoms.

In one preferred aspect X is a group selected from unsubstituted alkynyl groups.

In one preferred aspect X is a group selected unsubstituted C₆-C₂₄ alkynyl groups.

In one preferred aspect X is a group selected from unsubstituted alkynyl groups having a chain length of 6 to 24 atoms.

In one preferred aspect X is a group selected from unsubstituted C₁₀-C₁₈ alkynyl groups.

In one preferred aspect X is a group selected from unsubstituted alkynyl groups having a chain length of 10 to 18 atoms.

In one preferred aspect X is a group selected from unsubstituted C₁₆ or C₁₇ alkynyl groups.

In one preferred aspect X is a group selected from unsubstituted alkynyl having a chain length of 16 or 17 atoms.

In one preferred aspect X is a hydrocarbon chain. By “hydrocarbon chain” it is meant a linear hydrocarbon group.

When X contains one or more double bonds, preferably at least one, more preferably each is in cis configuration.

In one preferred aspect the C≡C of the acetylenic hydrocarbyl group is distanced from the terminal end of the acetylenic hydrocarbyl group by from 2 to 15 carbons.

In one preferred aspect the C≡C of the acetylenic hydrocarbyl group is distanced from the terminal end of the acetylenic hydrocarbyl group by 2 carbons.

In one preferred aspect the C≡C of the acetylenic hydrocarbyl group is distanced from the terminal end of the acetylenic hydrocarbyl group by 3 carbons.

In one preferred aspect the C≡C of the acetylenic hydrocarbyl group is distanced from the terminal end of the acetylenic hydrocarbyl group by 7 carbons.

In one preferred aspect the C≡C of the acetylenic hydrocarbyl group is distanced from the terminal end of the acetylenic hydrocarbyl group by 13 carbons.

Y

As discussed above Y is O or CH₂.

In one preferred aspect Y is CH₂.

In one preferred aspect when Y is CH₂, the chain X-Y-Z contains an even number of atoms. It will be understood that the chain length of X-Y-Z is the longest chain of directly bonded atoms within moiety X-Y-Z. It will be understood that a chain does not include atoms of cyclic substituents or substituents of a terminal carbon.

In one preferred aspect the chain X-Y-Z contains an even number of atoms.

Z

As discussed above Z is an optional hydrocarbyl group.

In one preferred aspect Z is an alkyl group.

In one preferred aspect Z is a C₁-C₁₀, preferably C₁-C₆, preferably C₁-C₃ alkyl group. Preferably Z is —CH₂—.

Compounds

In one aspect the compound is of the formula

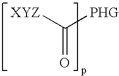

wherein p is at least 1, such as 1 to 10000, 1 to 1000, 1 to 100, 1 to 50, 1 to 20, 1 to 10, preferably 1 to 5, preferably 1, 2 or 3, and wherein each W, X, Y and Z is selected independently of each other.

Examples of suitable compounds from which the polar head group may be derived for given values of p are as follows

| p | |
|---|---|
| 1 | glycerols |
| | alcohols |
| | alkoxy compounds |
| | lysophospholipids |
| | monoacylglycerols |
| | gangliosides |
| | sphingomyelins |
| | cerebrosides |
| 2 | phosphatidylcholines (PC) |
| | phosphatidylethanolamines (PE), |
| | phosphatidylserines (PS) |
| | phosphatidylinositols (PI) |
| | diacylglycerols |
| | Phosphatidic acids |
| | glycerocarbohydrates |
| | phosphatidylglycerols |
| 3 | triacylglycerols |
| 1 or more | polyalcohols |

In one aspect the compound is of the formula

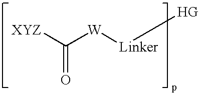

wherein p is 1 to 10, preferably 1 to 5, preferably 1, 2 or 3, and wherein each W, X, Y and Z is selected independently of each other.

In one aspect the compound is of the formula

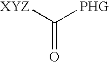

In one aspect the compound is of the formula

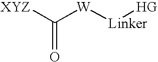

Preferably the compound comprises at least two non-polar moieties wherein each is independently selected from non-polar moieties of the formula X-Y-Z-.

In one preferred aspect the compound is of the formula

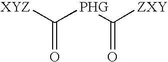

wherein each W, X, Y and Z is selected independently of each other.

In one preferred aspect the compound is of the formula

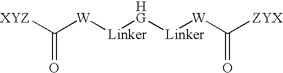

wherein each W, X, Y and Z is selected independently of each other.

In one aspect the compound comprises at least three non-polar moieties wherein each is independently selected from non-polar moieties of the formula X-Y-Z-.

In one preferred aspect the compound is of the formula

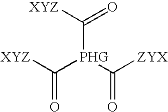

wherein each W, X, Y and Z is selected independently of each other.

In one preferred aspect the compound is of the formula

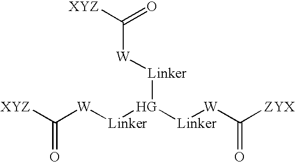

wherein each W, X, Y and Z is selected independently of each other.

In a preferred aspect the present invention provides a compound of the formula

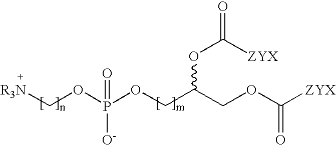

wherein R is independently selected from H and hydrocarbyl, n is from 1 to 10, m is from 1 to 10. In addition the present invention provides the compound

in admixture with or associated with a nucleotide sequence

for use in therapy.

for use in the manufacture of a medicament for the treatment of genetic disorder or condition or disease

a cationic liposome formed therefrom

a method of preparing a cationic liposome comprising forming the cationic liposome from the compound

the cationic liposome and use thereof

a pharmaceutical composition comprising the compound admixed with a pharmaceutical and, optionally, admixed with a pharmaceutically acceptable diluent, carrier or excipient

in S or R isomeric form, preferably in R isomeric form

Preferably -ZYX is a group of the formula CₚH₂ₚ₋₃ wherein p is from 3 to 30, preferably 10 to 25, preferably 15 to 20, preferably 15, 16, 17 or 18 carbon atoms.

Further highly preferred aspects of the present invention are described below. The present invention may provide

a compound is of the formula

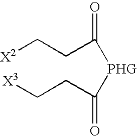

wherein X² and X³ are independently selected from unsubstituted C₁₀-C₁₈ alkynyl.

a compound is of the formula

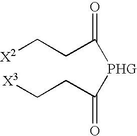

wherein X² and X³ are independently selected from unsubstituted C₁₄ alkynyl and unsubstituted C₁₅ alkynyl.

a compound is of the formula

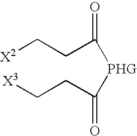

wherein X² and X³ are independently selected from CH₃(CH₂)₁₂C≡C—, CH₃CH₂CH₂C≡C(CH₂)₁₀—, CH₃(CH₂)₇C≡C(CH₂)₅—, CH₃(CH₂)₆C≡C(CH₂)₅—, and CH₃CH₂C≡C(CH₂)₁₀—.

a compound is of the formula

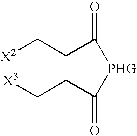

wherein X² and X³ are independently selected from unsubstituted C₁₀-C₁₈ alkynyl,

wherein the polar head group is derived from the polar head group of a phospholipid.

a compound is of the formula

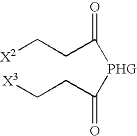

wherein X² and X³ are independently selected from unsubstituted C₁₄ alkynyl and unsubstituted C₁₅ alkynyl, wherein the polar head group is derived from the polar head group of a phospholipid.

a compound is of the formula

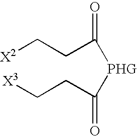

wherein X² and X³ are independently selected from CH₃(CH₂)₁₂C≡C—, CH₃CH₂CH₂C≡C(CH₂)₁₀—, CH₃(CH₂)₇C≡C(CH₂)₅—, CH₃(CH₂)₆C≡C(CH₂)₅—, and CH₃CH₂C≡C(CH₂)₁₀—, wherein the polar head group is derived from the polar head group of a phospholipid.

a compound is of the formula

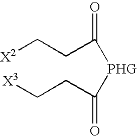

wherein X² and X³ are independently selected from unsubstituted C₁₀-C₁₈ alkynyl, wherein the polar head group is derived from the polar head group of a lipid selected from phosphatidylcholine (PC) phosphatidylethanolamine (PE), 3-N,N-dimethylaminopropan-1,2-diol (DAP) and 3-N,N,N-trimethylammoniopropan-1,2-diol (TAP).

a compound is of the formula

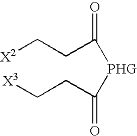

wherein x² and X³ are independently selected from unsubstituted C₁₄ alkynyl and unsubstituted C₁₅ alkynyl, wherein the polar head group is derived from the polar head group of a lipid selected from phosphatidylcholine (PC) phosphatidylethanolamine (PE), 3-N,N-dimethylaminopropan-1,2-diol (DAP) and 3-N,N,N-trimethylammoniopropan-1,2-diol (TAP).

a compound is of the formula

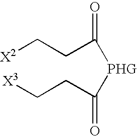

wherein X² and X³ are independently selected from CH₃(CH₂)₁₂C≡C—, CH₃CH₂CH₂C≡C(CH₂)₁₀—, CH₃(CH₂)₇C≡C(CH₂)₅—, CH₃(CH₂)₆C≡C(CH₂)₅—, and CH₃CH₂C≡C(CH₂)₁₀—, wherein the polar head group is derived from the polar head group of a lipid selected from phosphatidylcholine (PC) phosphatidylethanolamine (PE), 3-N,N-dimethylaminopropan-1,2-diol (DAP) and 3-N,N,N-trimethylammoniopropan-1,2-diol (TAP).

Further Aspects

Preferably the therapeutic agent of the composition is a nucleotide sequence. Preferably the compound is in admixture with or associated with a nucleotide sequence.

The nucleotide sequence may be part or all of an expression system that may be useful in therapy, such as gene therapy.

In a preferred aspect the compound of the present invention is in admixture with a condensed polypeptide/nucleic acid complex to provide a non-viral nucleic acid delivery vector. The condensed polypeptide/nucleic acid complex preferably include those disclosed in WO 01/48233. WO 01/48233 relates to a non-viral nucleic acid delivery vector comprising a condensed polypeptide/nucleic acid complex and a cationic lipid, wherein the complex comprises (a) a nucleic acid sequence of interest (NOI); and (b) one or more viral nucleic acid packaging polypeptides, or derivatives thereof, said polypeptides or derivatives thereof being (i) capable of binding to the NOI; and (ii) capable of condensing the NOI; and wherein the NOI is heterologous to the polypeptide.

Preferably the polypeptides or derivatives thereof are capable of binding to the nucleic acid complex. Preferably the polypeptides or derivatives thereof are capable of condensing the nucleic acid complex. Preferably the nucleic acid complex is heterologous to the polypeptides or derivatives thereof.

The compound of the present invention may used as partial or complete replacement of the cationic lipid of WO 01/48233. Thus in a preferred aspects the present invention provides

a non-viral nucleic acid delivery vector comprising a condensed polypeptide/nucleic acid complex, a cationic lipid and a compound in accordance with the present invention, wherein the complex comprises (a) a nucleic acid sequence of interest (NOI); and (b) one or more viral nucleic acid packaging polypeptides, or derivatives thereof, said polypeptides or derivatives thereof being (i) capable of binding to the NOI; and (ii) capable of condensing the NOI; and wherein the NOI is heterologous to the polypeptide.

a non-viral nucleic acid delivery vector comprising a condensed polypeptide/nucleic acid complex and a compound in accordance with the present invention, wherein the complex comprises (a) a nucleic acid sequence of interest (NOI); and (b) one or more viral nucleic acid packaging polypeptides, or derivatives thereof, said polypeptides or derivatives thereof being (i) capable of binding to the NOI; and (ii) capable of condensing the NOI; and wherein the NOI is heterologous to the polypeptide.

The compounds of the present invention may be combined with a liposome or formulated into micellar form to assist in administration.

In a further aspect the present compound maybe formulated in a cochleate delivery vehicles. Cochleate delivery vehicles represent a new technology platform for oral delivery of drugs. Cochleates are stable phospholipid-cation precipitates composed of simple, naturally occurring materials, for example, phosphatidylserine and calcium. Cochleates are a potential nanosized system that can encapsulate hydrophobic, amphiphilic, negatively or positively charged moieties.

In one aspect the compound of the present invention is an isolated form or purified form.

For example, the compound may be in a form or at a purity other than that found in a biological system such as in vivo.

The compounds of the present invention may be formulated to provide a pharmaceutical composition comprising a compound of the invention optionally admixed with a pharmaceutically acceptable carrier, diluent, excipient or adjuvant.

Pharmaceutical Composition

The present invention also provides a pharmaceutical composition comprising a therapeutically effective amount of the agent of the present invention and a pharmaceutically acceptable carrier, diluent or excipients (including combinations thereof).

This is a composition that comprises or consists of a therapeutically effective amount of a pharmaceutically active agent. It preferably includes a pharmaceutically acceptable carrier, diluent or excipients (including combinations thereof. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

This pharmaceutical composition will desirably be provided in a sterile form. It may be provided in unit dosage form and will generally be provided in a sealed container. A plurality of unit dosage forms may be provided.

Pharmaceutical compositions within the scope of the present invention may include one or more of the following: preserving agents, solubilising agents, stabilising agents, wetting agents, emulsifiers, sweeteners, colourants, flavouring agents, odourants, salts compounds of the present invention may themselves be provided in the form of a pharmaceutically acceptable salt), buffers, coating agents, antioxidants, suspending agents, adjuvants, excipients and diluents. Examples of preservatives include sodium benzoate, sorbic acid and esters of p-hydroxybenzoic acid.

They may also contain other therapeutically active agents in addition to compounds of the present invention. Where two or more therapeutic agents are used they may be administered separately (e.g. at different times and/or via different routes) and therefore do not always need to be present in a single composition. Thus combination therapy is within the scope of the present invention.

Route of Administration

A pharmaceutical composition within the scope of the present invention may be adapted for administration by any appropriate route. For example, it may be administered by the oral (including buccal or sublingual), rectal, nasal, topical (including buccal, sublingual or transdermal), vaginal or parenteral (including subcutaneous, intramuscular, intravenous or intradermal) routes. Such a composition may be prepared by any method known in the art of pharmacy, for example by admixing one or more active ingredients with a suitable carrier.

Different drug delivery systems can be used to administer pharmaceutical compositions of the present invention, depending upon the desired route of administration. Drug delivery systems are described, for example, by Langer (Science 249:1527-1533 (1991)) and by Illium and Davis (Current Opinions in Biotechnology 2: 254-259 (1991)). Different routes of administration for drug delivery will now be considered in greater detail:

The agents of the present invention may be administered alone but will generally be administered as a pharmaceutical composition—e.g. when the agent is in admixture with a suitable pharmaceutical excipient, diluent or carrier selected with regard to the intended route of administration and standard pharmaceutical practice.

For example, the agent can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, via the penis, vaginal, epidural, sublingual.

It is to be understood that not all of the agent need be administered by the same route. Likewise, if the composition comprises more than one active component, then those components may be administered by different routes.

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

(I) Oral Administration

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, maize starch or derivatives thereof, stearic acid or salts thereof. Soft gelatine capsules may comprise vegetable oils, waxes, fats, semi-solid, or liquid polyols etc. Solutions and syrups may comprise water, polyols and sugars. For the preparation of suspensions oils (e.g. vegetable oils) may be used to provide oil-in-water or water-in-oil suspensions. An active agent intended for oral administration may be coated with or admixed with a material that delays disintegration and/or absorption of the active agent in the gastrointestinal tract (e.g. glyceryl monostearate or glyceryl distearate may be used). Thus the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

(II) Transdermal Administration

Pharmaceutical compositions adapted for transdermal administration may be provided as discrete patches intended to remain in intimate contact with the epidermis of the recipient for a prolonged period of time. For example, the active ingredient may be delivered from the patch by iontophoresis. (Iontophoresis is described in Pharmaceutical Research, 3(6:318 (1986).)

(III) Topical Administration

Alternatively, the agent of the present invention can be administered in the form of a suppository or pessary, or it may be applied topically in the form of a gel, hydrogel, lotion, solution, cream, ointment or dusting powder. The agent of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

(IV) Rectal Administration

Pharmaceutical compositions adapted for rectal administration may be provided as suppositories or enemas.

(V) Nasal Administration

Pharmaceutical compositions adapted for nasal administration may use solid carriers, e.g. powders (preferably having a particle size in the range of 20 to 500 microns). Powders can be administered in the manner in which snuff is taken, i.e. by rapid inhalation through the nose from a container of powder held close to the nose. Compositions adopted for nasal administration may alternatively use liquid carriers, e.g. nasal sprays or nasal drops. These may comprise aqueous or oil solutions of the active ingredient.

Compositions for administration by inhalation may be supplied in specially adapted devices—e.g. in pressurised aerosols, nebulizers or insufflators. These devices can be constructed so as to provide predetermined dosages of the active ingredient.

(VI) Vaginal Administration

Pharmaceutical compositions adapted for vaginal administration may be provided as pessaries, tampons, creams, gels, pastes, foams or spray formulations.

(VII) Parenteral Administration

If the agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Transdermal

“Transdermal” refers to the delivery of a compound by passage through the skin and into the blood stream.

Transmucosal

“Transmucosal” refers to delivery of a compound by passage of the compound through the mucosal tissue and into the blood stream.

Transurethral or Intraurethral

“Transurethral” or “intraurethral” refers to delivery of a drug into the urethra, such that the drug contacts and passes through the wall of the urethra and enters into the blood stream.

Penetration Enhancement or Permeation Enhancement

“Penetration enhancement” or “permeation enhancement” refers to an increase in the permeability of the skin or mucosal tissue to a selected pharmacologically active compound such that the rate at which the compound permeates through the skin or mucosal tissue is increased.

Penetration enhancers may include, for example, dimethylsulfoxide (DMSO), dimethyl fbrmamide (DMF),N,N-dimethylacetamide (DMA), decylmethylsulfoxide (CIOMSO), polyethyleneglycol monolaurate (PEGML), glyceral monolaurate, lecithin, 1-substituted azacycloheptanones, particularly 1-N-dodecylcyclazacylcoheptanones (available under the trademark Azone TM from Nelson Research & Development Co., Irvine, Calif.), alcohols and the like.

Carriers or Vehicles

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

Examples of pharmaceutically acceptable carriers include, for example, water, salt solutions, alcohol, silicone, waxes, petroleum jelly, vegetable oils, polyethylene glycols, propylene glycol, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Epidermal Drug Delivery (Transfersomes)

Transfersomes (“carrying bodies”) are complex, most often vesicular, bi- or multi-component aggregates capable of crossing barriers and of transferring material between the application and the destination sites. Transfersomes are sold by IDEA Corporation, Munich, Germany, and TRANSFERSOME is a trade mark of that company. Transfersome transdermal drug delivery technology may be used for controllable and non-invasive delivery of a wide variety of large molecules as well as for the improved delivery of small molecules, including the metabolic enzyme antagonists and/or drugs of the present invention.

Transfersomes may be optimised to attain extremely flexible and self-regulating membranes. They are therefore deformable and consequently can cross microporous barriers efficiently, even when the available passages are much smaller than the average aggregate size. Transfersome formulations are typically composed of natural amphipatic compounds suspended in a water-based solution, optionally containing biocompatible surfactants. Vesicular Transfersomes consist of a lipid bilayer surrounding an aqueous core and further contain at least one component, capable of softening the membrane. The bilayer of a Transferosome is therefore more flexible than a liposome membrane, even metastable. Transfersome vesicles consequently change their shape easily by adjusting locally to ambient stress.

Skin is one of the best biological barriers. Its outermost part, the horny layer, reaches less than 10% into the depth of the skin but contributes over 80% to the skin permeability barrier. This body protecting layer consists of overlapping, flaccid corneocytes, organized in columnar clusters, sealed with multilamellar lipid sheets that are covalently attached to the cell membranes and very tightly packed. Generally, the average number of and the degree of order in the intercellular lipid lamellae increases toward the skin surface. This is accompanied by a continuous, but nonlinear, decrease in local water content near the surface. Notwithstanding this, the peak skin barrier is located in the inner half of the horny layer, where the intercellular lipid seals are already formed, but not yet compromised by the skin cells detachment.

Passage of fransfersome aggregates across the skin is a function of vesicle membrane flexibility, hydrophilicity, and the ability to retain vesicle integrity, while the aggregate undergoes a significant change in shape. When a suspension of Transfersome vesicles is placed on the surface of the skin, water evaporates from the relatively arid skin surface and the vesicles start to dry out. Due to the strong polarity of major Transfersome ingredients, the large number of hydrophilic groups on the membrane, assisted by the softness of the membrane, the vesicles are attracted to the areas of higher water content in the narrow gaps between adjoining cells in the skin barrier, enabling skin penetration of the vehicle. This, together with the vesicle's extreme ability to deform, enables Transfersome aggregates to open, temporarily, the tiny “cracks” through which water normally evaporates out of the skin. Channels between the skin cells, two orders of magnitude wider than the original micropores, are thus created. Such newly activated passages can accommodate sufficiently deformable vesicles, which maintain their integrity but change their shape to fit the channel. Along the resulting “virtual pathways”, or “Virtual channels” in the horny layer, Transfersomes reach regions of high water content in the deeper skin layers. There, the vesicles (re)distribute. Since Transfersomes are too large to enter the blood vessels locally, they bypass the capillary bed and get to subcutaneous tissue, where they accumulate.

Although small molecules that have crossed the horny layer of the skin (stratum corneum) are normally cleared from the skin through the blood circulation, delivery of drugs by means of Transfersome vesicles allows accumulation of drug deep under the skin. Due to their large size, the vesicles are cleared slowly from the skin and associated drugs can accumulate at the site. Transfersome mediated administration of weight drugs, consequently, tends to shift the drug distribution towards the deep tissue under the application site.

Blood Brain Barrier (BBB)

Pharmaceutical compositions may be designed to pass across the blood brain barrier (BBB). For example, a carrier such as a fatty acid, inositol or cholesterol may be selected that is able to penetrate the BBB. The carrier may be a substance that enters the brain through a specific transport system in brain endothelial cells, such as insulin-like growth factor I or II. The carrier may be coupled to the active agent or may contain/be in admixture with the active agent. Liposomes can be used to cross the BBB. WO91/04014 describes a liposome delivery system in which an active agent can be encapsulated/embedded and in which molecules that are normally transported across the BBB (e.g. insulin or insulin-like growth factor I or II) are present on the liposome outer surface. Liposome delivery systems are also discussed in U.S. Pat. No. 4,704,355.

Polymer Delivery/Therapeutics

The agents may further be delivered attached to polymers. Polymer based therapeutics have been proposed to be effective delivery systems, and generally comprise one or more agents to be delivered attached to a polymeric molecule, which acts as a carrier. The agents are thus disposed on the polymer backbone, and are carried into the target cell together with the polymer.

The agents may be coupled, fused, mixed, combined, or otherwise joined to a polymer. The coupling, etc between the agent and the polymer may be permanent or transient, and may involve covalent or non-covalent interactions (including ionic interactions, hydrophobic forces, Van der Waals interactions, etc). The exact mode of coupling is not important, so long as the agent is taken into a target cell substantially together with the polymer. For simplicity, the entity comprising the agent attached to the polymer carrier is referred to here as a “polymer-agent conjugate”.

Any suitable polymer, for example, a natural or synthetic polymer, may be used, preferably the carrier polymer is a synthetic polymer such as PEG. More preferably, the carrier polymer is a biologically inert molecule. Particular examples of polymers include polyethylene glycol (PEG), N-(2-hydroxypropyl) methacrylamide (HPMA) copolymers, polyamidoamine (PAMAM) dendrimers, HEMA, linear polyamidoamine polymers etc. Any suitable linker for attaching the agent to the polymer may be used. Preferably, the linker is a biodegradable linker. Use of biodegradable linkers enables controlled release of the agent on exposure to the extracellular or intracellular environment. High molecular weight macromolecules are unable to diffuse passively into cells, and are instead engulfed as membrane-encircled vesicles. Once inside the vesicle, intracellular enzymes may act on the polymer-agent conjugate to effect release of the agent. Controlled intracellular release circumvents the toxic side effects associated with many drugs.

Furthermore, agents may be conjugated, attached etc by methods known in the art to any suitable polymer, and delivered. The agents may in particular comprise any of the molecules referred to as “second agents”, such as polypeptides, nucleic acids, macromolecules, etc, as described in the section below. In particular, the agent may comprise a pro-drug as described elsewhere.

The ability to choose the starting polymer enables the engineering of polymer-agent conjugates for desirable properties. The molecular weight of the polymer (and thus the polymer-agent conjugate), as well as its charge and hydrophobicity properties, may be precisely tailored. Advantages of using polymer-agent conjugates include economy of manufacture, stability (longer shelf life) and reduction of immunogencity and side effects. Furthermore, polymer-agent conjugates are especially useful for the targeting of tumour cells because of the enhanced permeability and retention (EPR) effect, in which growing tumours are more ‘leaky’ to circulating macromolecules and large particules, allowing them easy access to the interior of the tumour. Increased accumulation and low toxicity (typically 10-20% of the toxicity of the free agent) are also observed. Use of hyperbranched dendrimers, for example, PAMAM dendrimers, is particularly advantageous in that they enable monodisperse compositions to be made and also flexibility of attachment sites (within the interior or the exterior of the dendrimer). The pH responsiveness of polymer-agent conjugates, for example, those conjugated to polyamindoamine polymers, may be tailored for particular intracellular environments. This enables the drug to be released only when the polymer therapeutic encounters a particular pH or range of pH, i.e., within a particular intracellular compartment. The polymer agent conjugates may further comprise a targeting means, such as an immunoglobulin or antibody, which directs the polymer-agent conjugate to certain tissues, organs or cells comprising a target, for example, a particular antigen. Other targeting means are described elsewhere in this document, and are also known in the art.

Particular examples of polymer-agent conjugates include “Smancs”, comprising a conjugate of styrene-co-maleic anhydride and the antitumour protein neocarzinostatin, and a conjugate of PEG (poly-ethylene glycol) with L-asparaginase for treatment of leukaemia; PK1 (a conjugate of a HPMA copolymer with the anticancer drug doxorubicin); PK2 (similar to PK1, but furthermore including a galactose group for targeting primary and secondary liver cancer); a conjugate of HPMA copolymer with the anticancer agent captothecin; a conjugate of HPMA copolymer with the anticancer agent paclitaxel; HPMA copolymer-platinate, etc. Any of these polymer-agent conjugates are suitable for co-loading into the transgenic cells of the present invention.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The agent and/or the pharmaceutical composition of the present invention may be administered in accordance with a regimen of from 1 to 10 times per day, such as once or twice per day.

For oral and parenteral administration to human patients, the daily dosage level of the agent may be in single or divided doses.

Depending upon the need, the agent may be administered at a dose of from 0.01 to 30 mg/kg body weight, such as from 0.1 to 10 mg/kg, more preferably from 0.1 to 1 mg/kg body weight. Naturally, the dosages mentioned herein are exemplary of the average case. There can, of course, be individual instances where higher or lower dosage ranges are merited.

Therapeutically Effective Amount

“Therapeutically effective amount” refers to the amount of the therapeutic agent which is effective to achieve its intended purpose. While individual patient needs may vary, determination of optimal ranges for effective amounts of each nitric oxide adduct is within the skill of the art. Generally the dosage regimen for treating a condition with the compounds and/or compositions of this invention is selected in accordance with a variety of factors, including the type, age, weight, sex, diet and medical condition of the patient, the severity of the dysfunction, the route of administration, pharmacological considerations such as the activity, efficacy, pharmacokinetic and toxicology profiles of the particular compound used, whether a drug delivery system is used, and whether the compound is administered as part of a drug combination and can be adjusted by one skilled in the art. Thus, the dosage regimen actually employed may vary widely and therefore may deviate from the preferred dosage regimen set forth herein.

Individual

As used herein, the term “individual” refers to vertebrates, particularly members of the mammalian species. The term includes but is not limited to domestic animals, sports animals, primates and humans.

Pharmaceutical Combinations

In general, the agent may be used in combination with one or more other pharmaceutically active agents. The other agent is sometimes referred to as being an auxiliary agent.

Patient

“Patient” refers to animals, preferably mammals, more preferably humans.

Pharmaceutically Acceptable Salt

The agent may be in the form of—and/or may be administered as—a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Typically, a pharmaceutically acceptable salt may be readily prepared by using a desired acid or base, as appropriate. The salt may precipitate from solution and be collected by filtration or may be recovered by evaporation of the solvent.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

Disease States

The compound or composition of the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of bums, ulcers and periodontal disease and neurodegeneration; inhibition or activation of follicle-stimulating, hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebr, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

Treatment

This includes any therapeutic application that can benefit a human or non-human animal. The treatment of mammals is particularly preferred. Both human and veterinary treatments are within the scope of the present invention.

Treatment may be in respect of an existing condition or it may be prophylactic. It may be of an adult, a juvenile, an infant, a foetus, or a part of any of the aforesaid (e.g. an organ, tissue, cell, or nucleic acid molecule).

An active agent for use in treatment can be administered via any appropriate route and at any appropriate dosage. Dosages can vary between wide limits, depending upon the nature of the treatment, the age and condition of the individual to be treated, etc. and a physician will ultimately determine appropriate dosages to be used. However, without being bound by any particular dosages, a daily dosage of a compound of the present invention of from 1 μg to 1 mg/kg body weight may be suitable. The dosage may be repeated as often as appropriate. If side effects develop, the amount and/or frequency of the dosage can be reduced, in accordance with good clinical practice.

Polymorphic Form(S)/Asymmetric Carbon(S)

The agent of the present invention may exist in polymorphic form.

The agent of the present invention may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of a compound of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

Isotonic Variations

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Pro-Drug

It will be appreciated by those skilled in the art that the agent of the present invention may be derived from a prodrug. Examples of prodrugs include entities that have certain protected group(s) and which may not possess pharmacological activity as such, but may, in certain instances, be administered (such as orally or parenterally) and thereafter metabolised in the body to form the agent of the present invention which are pharmacologically active.

Pro-Moiety

It will be further appreciated that certain moieties known as “pro-moieties”, for example as described in “Design of Prodrugs” by H. Bundgaard, Elsevier, 1985 (the disclosure of which is hereby incorporated by reference), may be placed on appropriate functionalities of the agents. Such prodrugs are also included within the scope of the invention.

Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

In one embodiment of the present invention, the agent may be a chemically modified agent.

The chemical modification of an agent of the present invention may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

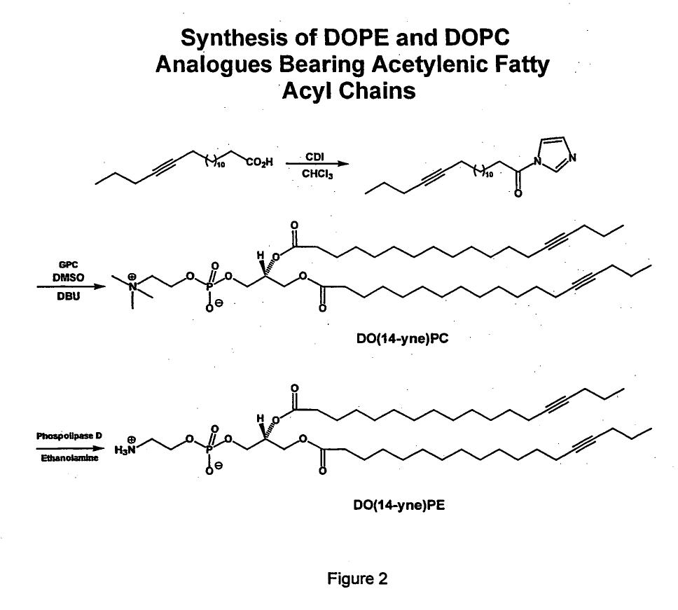

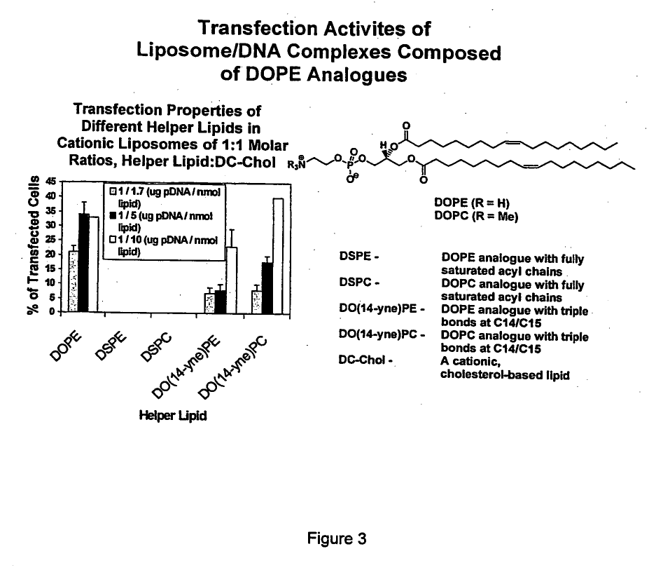

The present invention will now be described in further detail by way of example only with reference to the accompanying figures in which:—FIG. 1 shows a schematic; FIG. 2 shows a scheme; FIG. 3 shows a structure and graph;

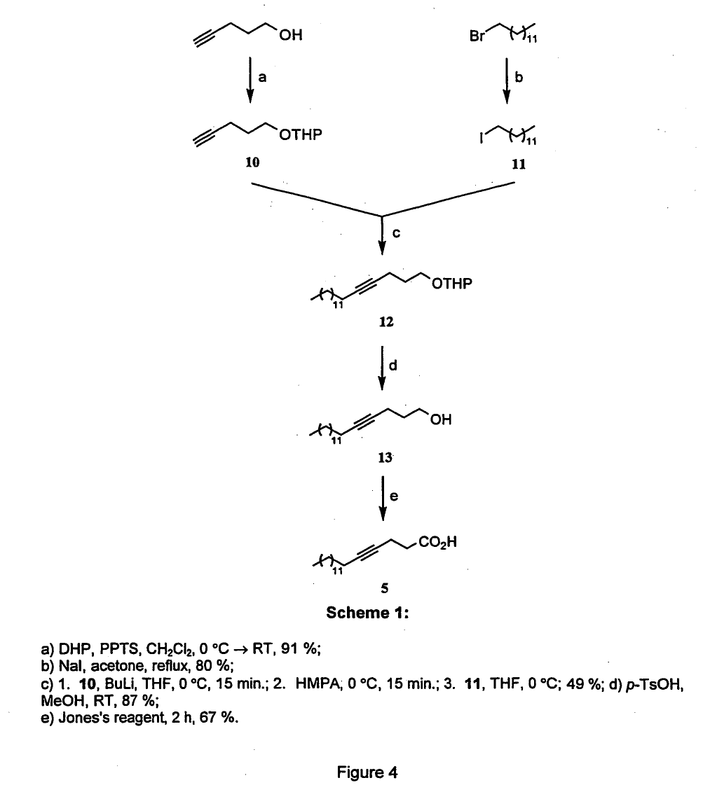

FIG. 4 shows a scheme;

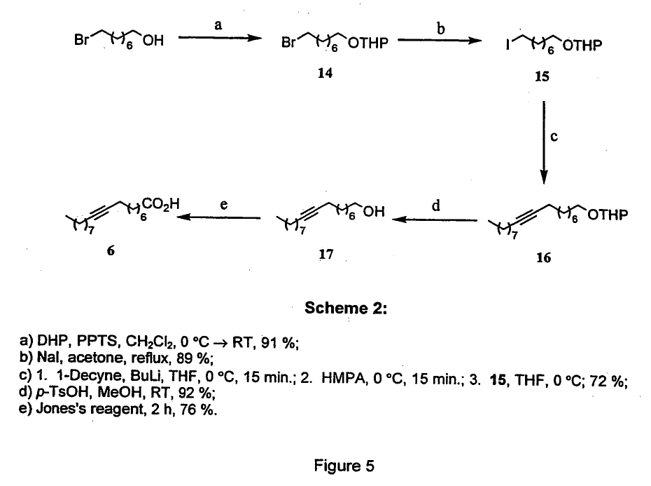

FIG. 5 shows a scheme;

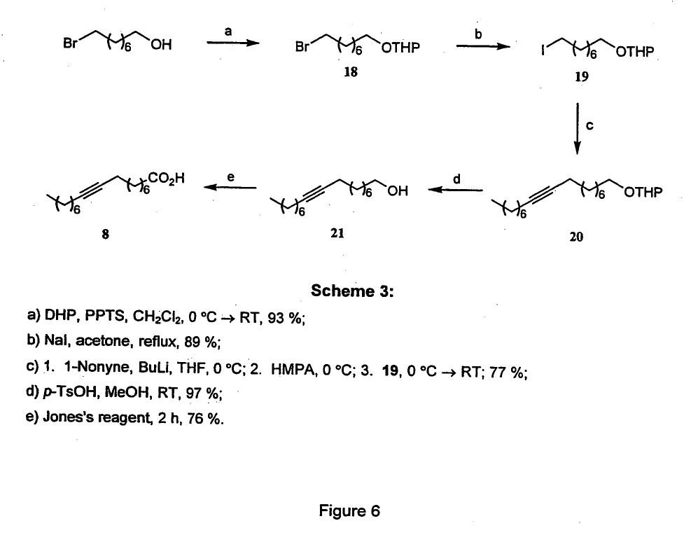

FIG. 6 shows a scheme;

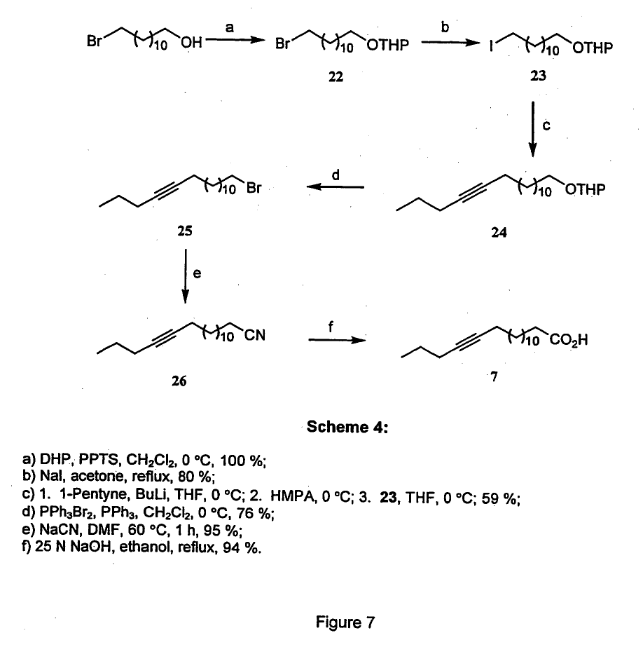

FIG. 7 shows a scheme;

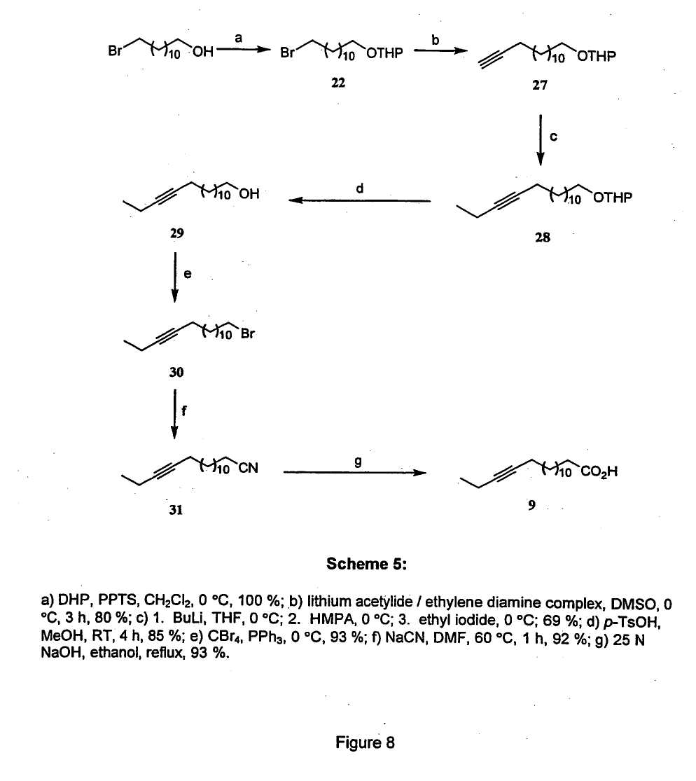

FIG. 8 shows a scheme;

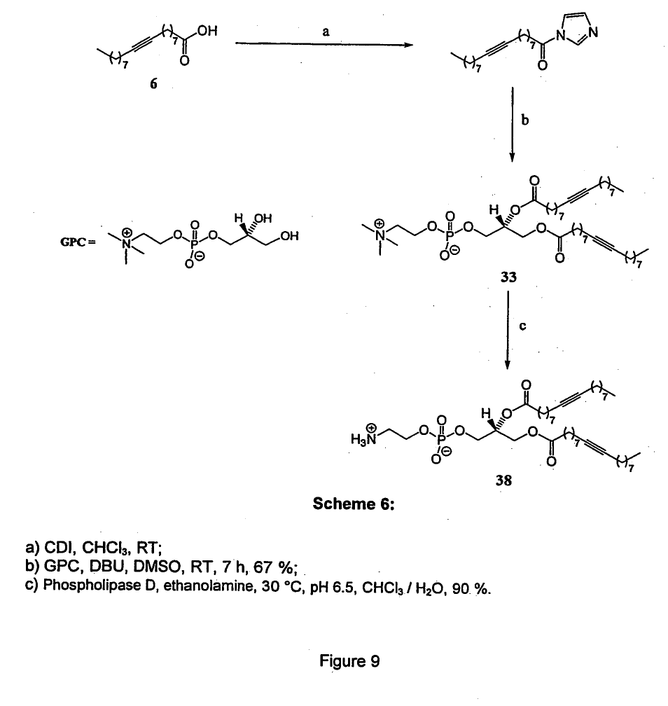

FIG. 9 shows a scheme;

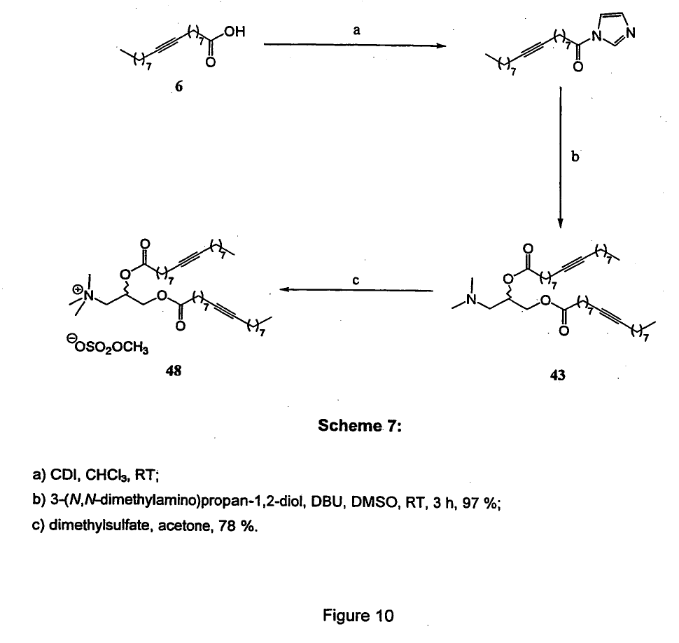

FIG. 10 shows a scheme;

FIG. 11 shows a graph;

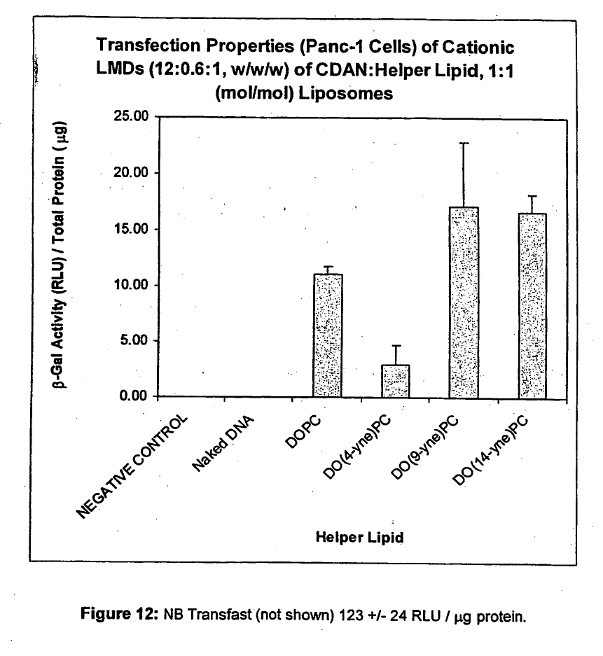

FIG. 12 shows a graph;

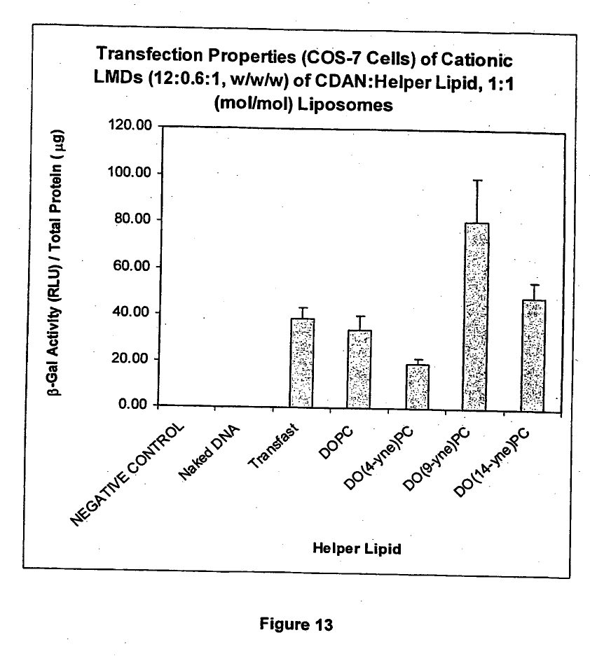

FIG. 13 shows a graph;

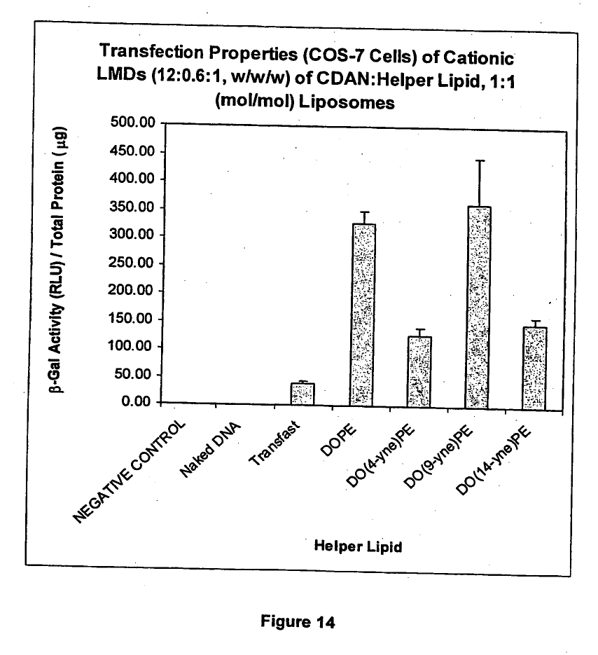

FIG. 14 shows a graph;

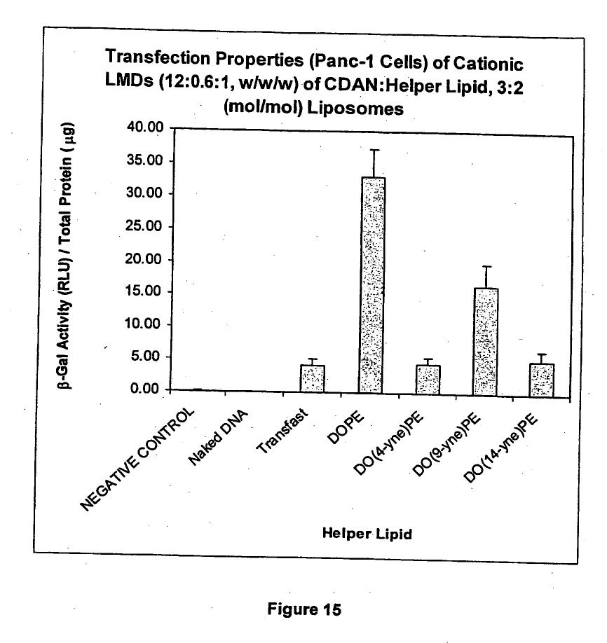

FIG. 15 shows a graph;

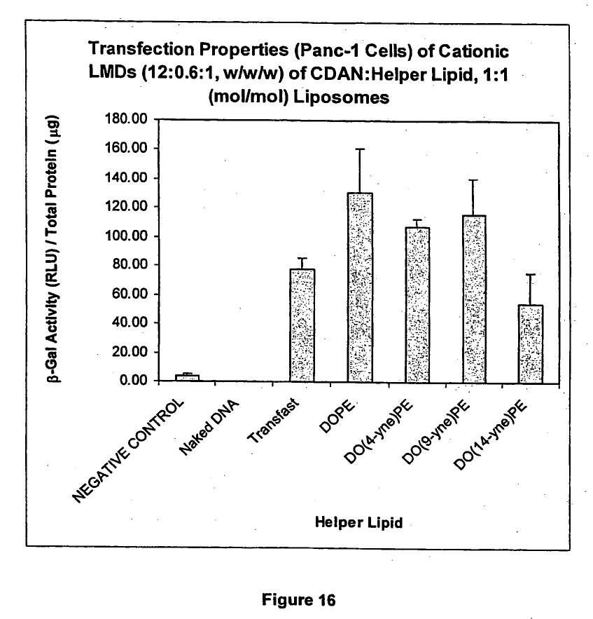

FIG. 16 shows a graph;

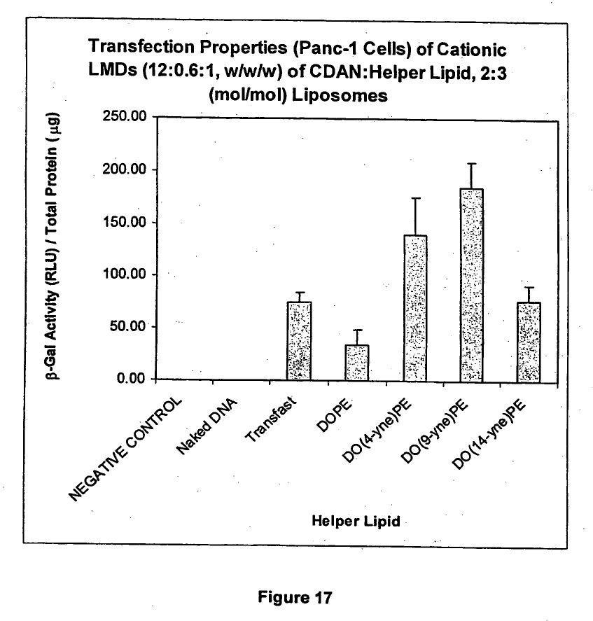

FIG. 17 shows a graph;

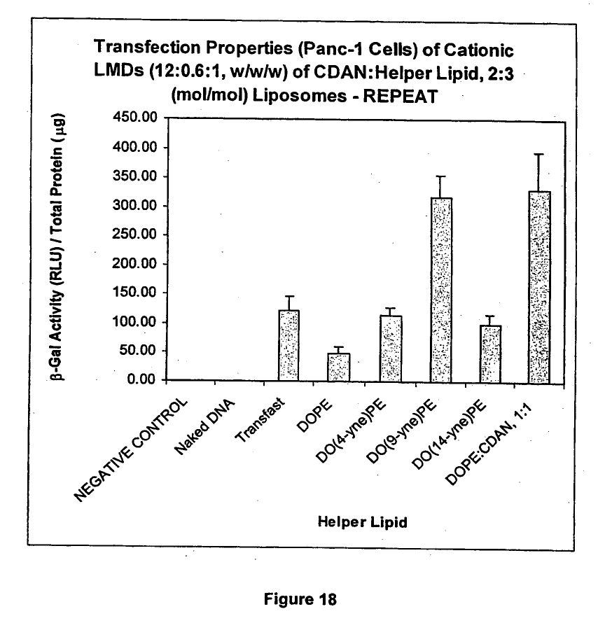

FIG. 18 shows a graph;

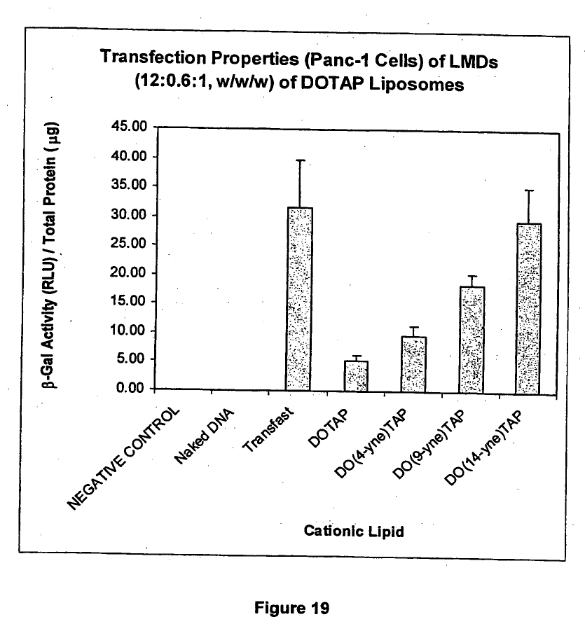

FIG. 19 shows a graph;

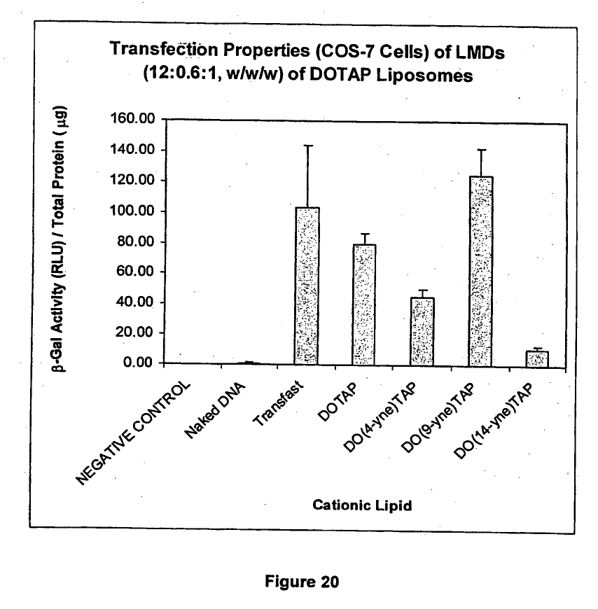

FIG. 20 shows a graph;

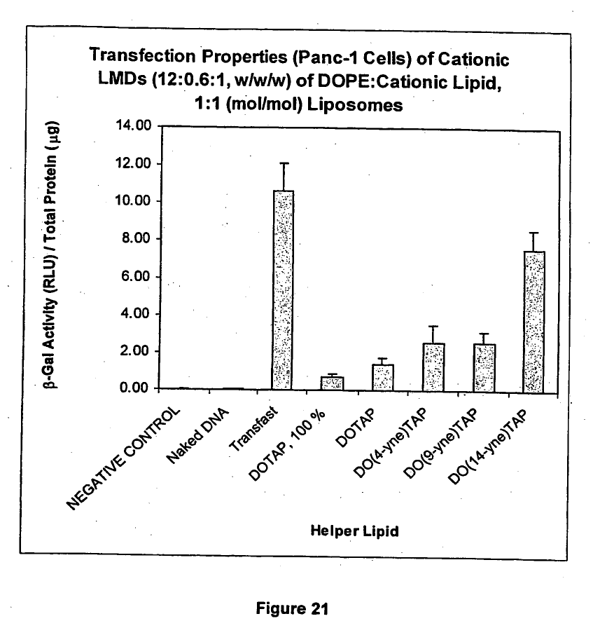

FIG. 21 shows a graph;

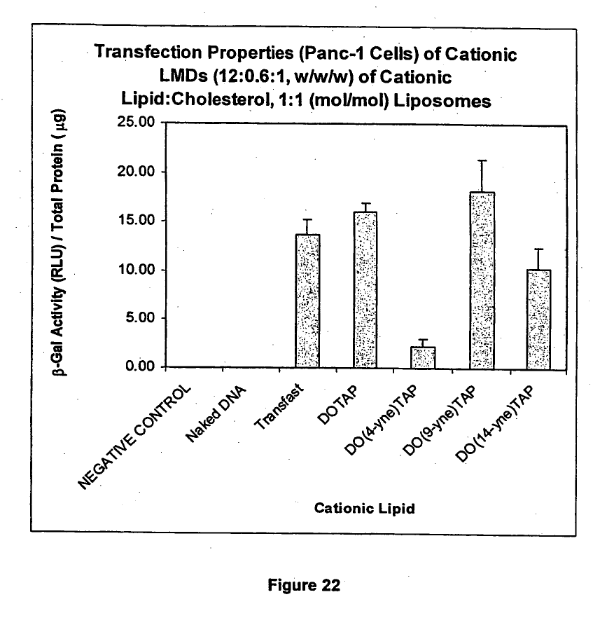

FIG. 22 shows a graph;

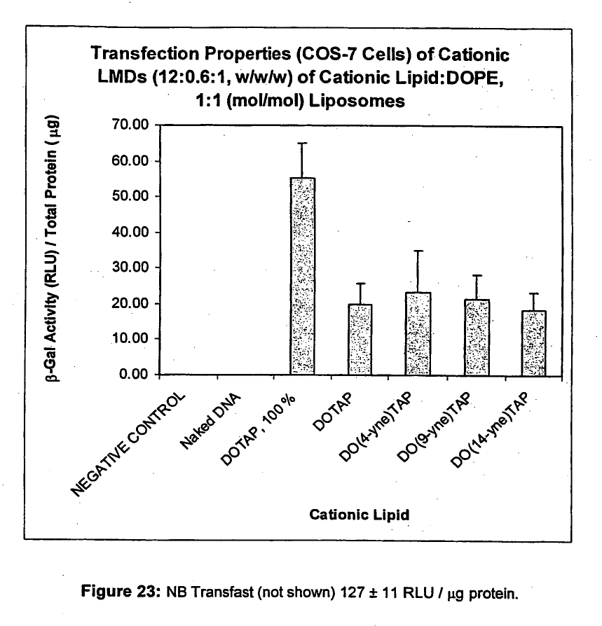

FIG. 23 shows a graph; and

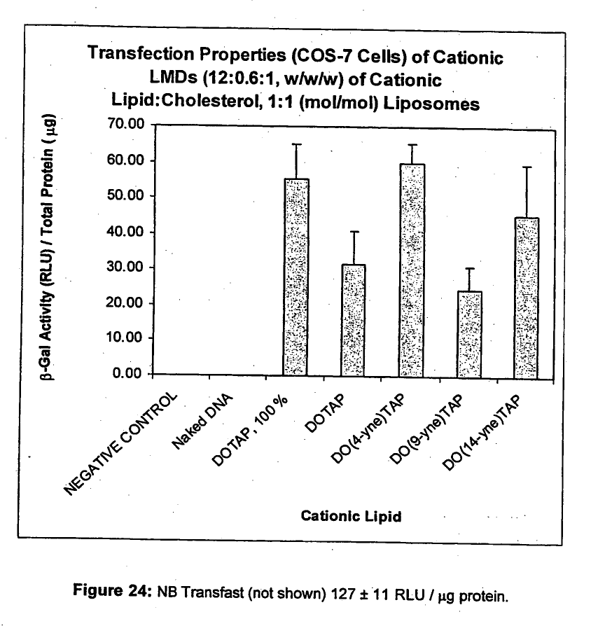

FIG. 24 shows a graph.

### EXAMPLE

### Example 1

DOPE, DSPE and DSPC were purchased directly from Sigma-Aldrich, Poole, Dorset, UK; DO(14-yne)PE and DO(14-yne)PC were synthesised as described in Scheme 4—FIG. 7 and FIG. 2. The transfection properties of these compounds in cationic liposomes of 1:1 molar ratios, helper lipid:DC-Chol were measured. The data from these studies are represented graphically in FIG. 9.

Syntheses of Monoacetylenic Fatty Acids

Five monoacetylenic fatty acids have been synthesised (see below): three C18 fatty acids (5, 6 and 7), differing in the position of the triple bond (4, 9 and 14, respectively) and two C17 fatty acids (8 and 9, triple bond in positions 9 and 14, respectively).

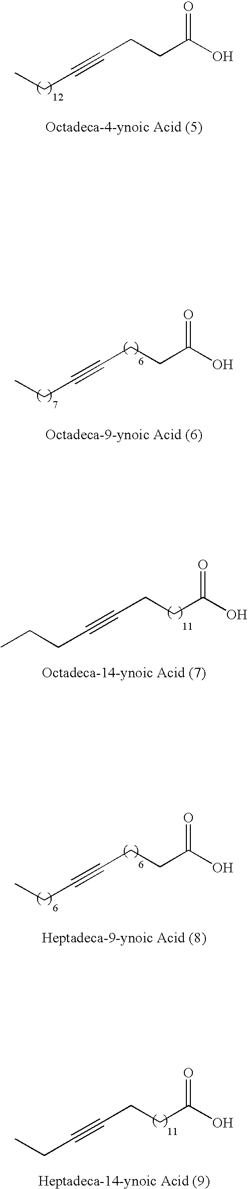

Monoacetylenic analogues of each of the following lipids were then prepared with the five fatty acids:

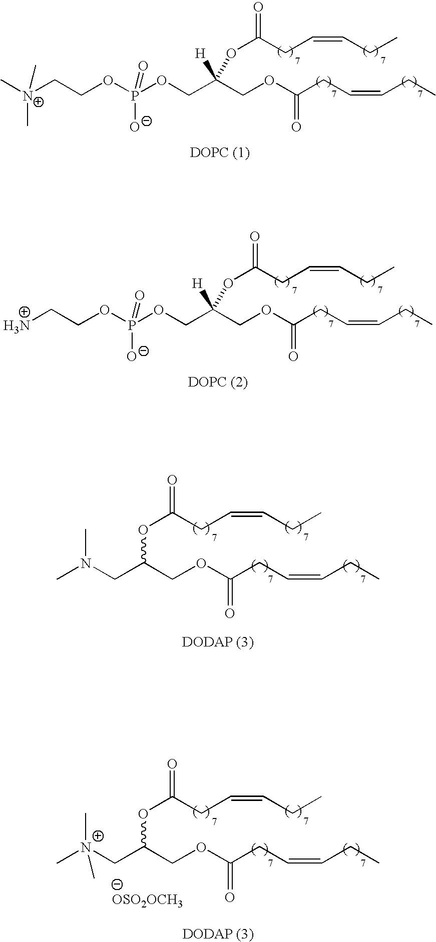

This provided us with a series of twenty lipids in total.

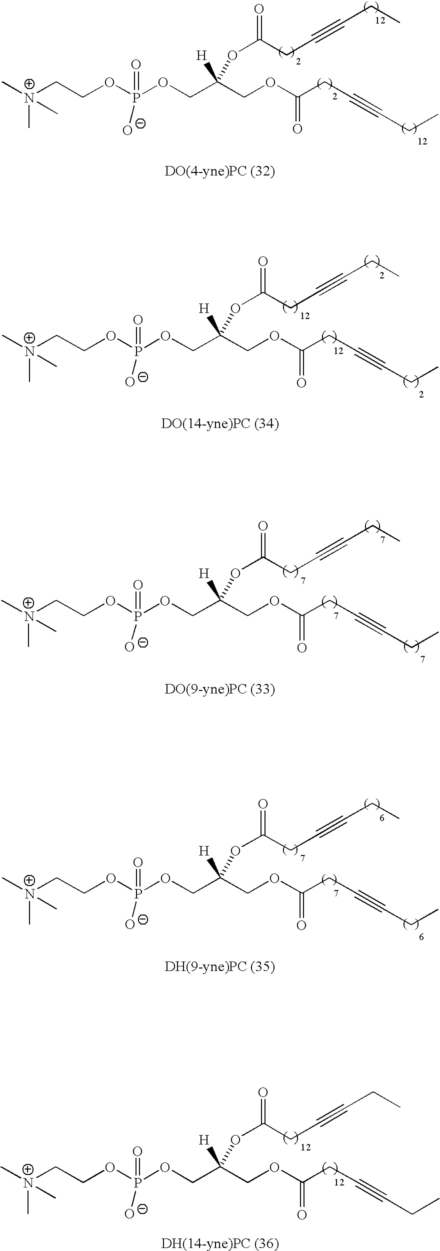

Five monoacetylenic DOPC-analogues.

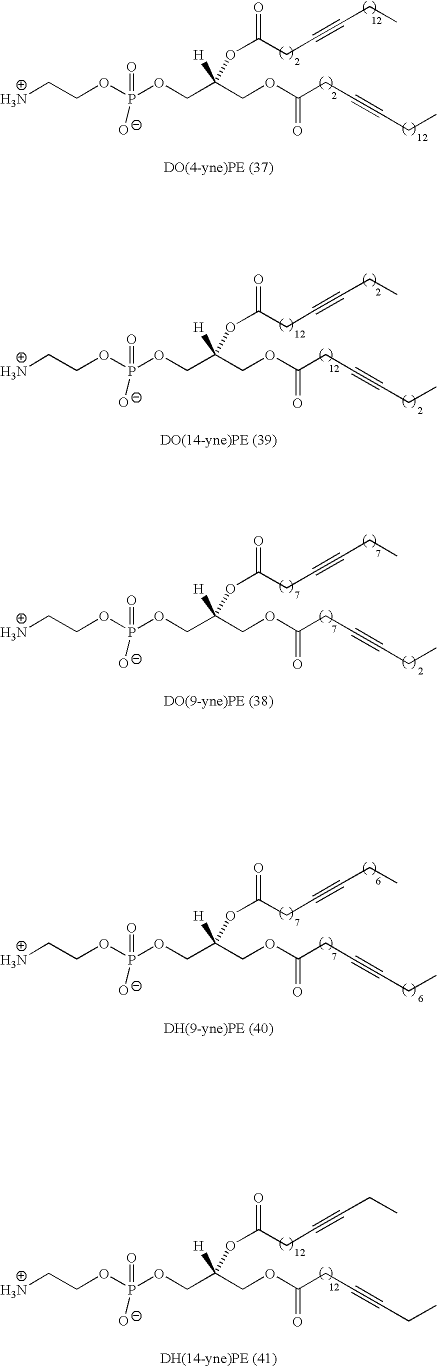

Five monoacetylenic DOPE-analogues

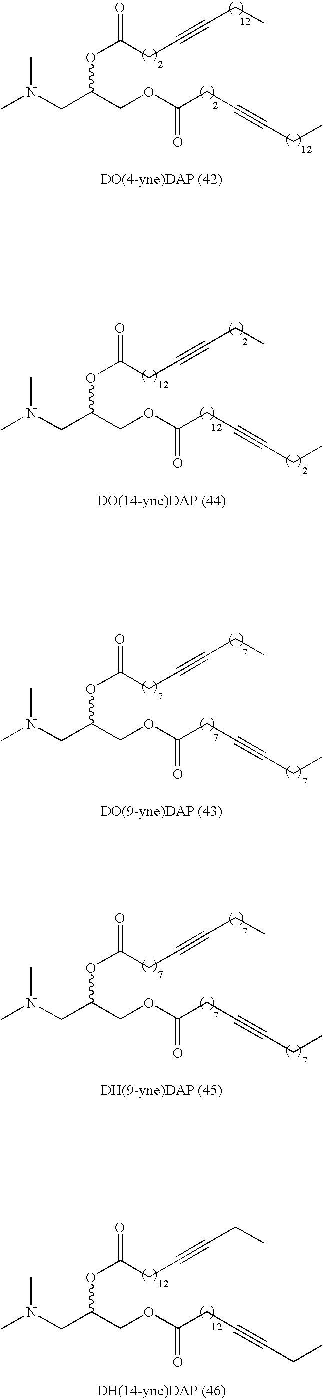

Five monoacetylenic DODAP-analogues.

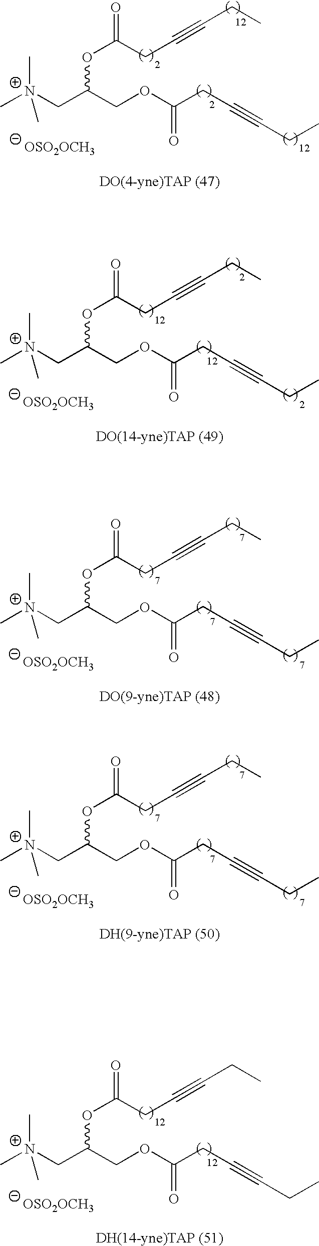

Five monoacetylenic DOTAP-analogues.

Syntheses of Octadeca-4-ynoic Acid (5), Octadeca-9-ynoic Acid (6) and Heptadeca-9-ynoic Acid (8)

Octadeca-4-ynoic acid (5) was prepared as follows (Scheme 1—FIG. 4). First, the THP-protected derivative of pent4-yn-1-ol (10) was generated by reaction with DHP under mild acid catalysis with PPTS in 91% yield. Meanwhile, 1-bromotridecane was converted to its more reactive iodo-analogue (11; 80% yield) by the well-known Finkelstein halogen exchange reaction.

Deprotonation of terminal alkyne 10 by BuLi in the presence of HMPA created the alkyne anion needed for the S_{N}2 reaction on 1-iodotridecane. This step proceeded in moderate yield (49%). Acid-catalysed hydrolysis of 12 unmasked the primary alcohol group which was subsequently oxidised with Jones's reagent (CrO₃, c.H₂SO₄) in acetone, affording octadeca4-ynoic acid (5) in 69% yield.

Octadeca-9-ynoic acid was synthesised similarly (Scheme 2—FIG. 5). First, the hydroxy group of 8-bromo-octan-1-ol was protected as the THP-ether using DHP and PPTS in almost quantitative yield (91%). The coupling of 1-decyne to more reactive 15 (cf 14) gave the internal alkyne product (16) in good yield (72%). THP-hydrolysis with TsOH in MeOH afforded alkynol 17 in 92% yield, then oxidation using Jones's reagent furnished acid 6 as a white powder (76%).

The synthesis of heptadeca-9-ynoic acid (8; Scheme 3—FIG. 6) was accomplished in an almost identical manner to that of octadeca-9-ynoic acid, differing only in the use of 1-nonyne as the reagent to introduce the internal C—C triple bond (as opposed to 1-decyne).

Syntheses of Ocatdeca-14-ynoic Acid (7) and Heptadeca-14-ynoic Acid (9)

As in the previous synthesis sub-section, the starting material for the preparation of ocatdeca-14-ynoic acid (7) was 12-bromododecan-1-ol (Scheme 4—FIG. 7). THP-protection of the primary alcohol group proceeded quantitatively, giving 22. Again, the Finkelstein exchange reaction was employed to generate the more reactive iodo-analogue (23).

Pent-1-yne was reacted with 23 under our improved alkyne deprotonation-alkylation procedure to give 24 in good yield (59%). The THP-ether functionality was converted directly into the bromide (25) with PPh₃Br₂/PPh₃ (76%); the resulting bromo-alkyne (25) was then subjected to an SN² reaction with CN⁻ to afford fatty acyl chain homologation. Forcing basic hydrolysis of the nitrile group (26) led to the introduction of the carboxylate group, affording octadeca-14-ynoic acid (7) in excellent yield (94%).

Heptadeca-14-ynoic acid (9) was prepared in an almost analogous fashion (Scheme 5—FIG. 8). The only differences were the need to introduce the internal alkyne functionality in two steps (as opposed to one step), due to the: availability of starting materials, and the THP-ether functionality was converted to the bromide in two steps (acid-catalysed hydrolysis liberated the primary alcohol group which then underwent an S_{N}2 reaction with CBr₄ and PPh₃ to give the desired bromide).

Syntheses of DOPC-, DOPE-, DODAP- and DOTAP-Analogues

The syntheses of the DOPC-analogues (e.g. 33) involved activation of the fatty acids (e.g. 6) as the acyl imidazolides, then reaction with snglycero-3-phosphocholine (GPC) in the presence of DBU. Yields averaged at around 60%. The syntheses of the DOPE-analogues (e.g. 38) were accomplished by a bi-phasic (chloroform/water) enzymatic transphosphatidylation of the DOPC-analogues with ethanolamine in yields of around 90% (Scheme 6—FIG. 9).

In a similar manner, the DODAP-analogues (e.g. 43) were synthesised in quantitative yields. Treatment of the DODAP compounds with dimethyl sulfate afforded the DOTAP-analogues (e.g. 48) as the methyl sulfate salts, in yields of between 70 and 80%. (Scheme 7—FIG. 10).

Transfection Data

Introduction

The monoacetylenic analogues of DOPC, DOPE, DODAP and DOTAP were synthesised as described above. Standard lipids for comparisons (DOPC, DOPE and DOTAP (chloride salt)) were purchased from Sigma-Aldrich, Poole, Dorset, UK. Our invention the cationic, cholesterol-based lipid N¹-cholesteryloxycarbonyl-3,7-diazanononane-1,9-diamine (CDAN)—net charge=+1.6 at pH 7.4.CDAN (below) was synthesised by our group previously,²² and is now available in a 1:1 molar ratio formulation with DOPE as Trojene™ (Avanti Polar Lipids, Inc., Alabaster, Ala., USA).

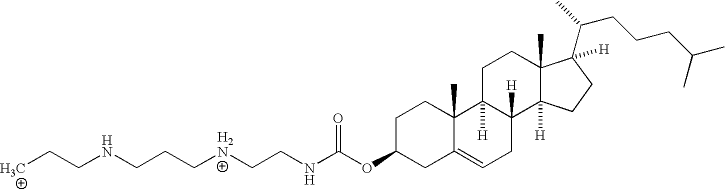

Purity of the lipids was checked by TLC or HPLC. All lipids were stored as stock solutions in anhydrous CH₂Cl₂, at concentrations of either 5 mg/ml or 10 mg/ml, at −80° C., under Ar. In order to prepare cationic liposomes, lipids were added to a round-bottomed flask, via syringe, under Ar, and further freshly distilled CH₂Cl₂ was added, if necessary, to give a concentration of around 2.5 mg/ml. Then, 4 mM HEPES pH 7.0 (1 ml) was added, and the biphasic system was swirled to mix. A liposomal suspension was created by removing the organic solvent under reduced pressure at 25° C., followed by sonication for 2-5 min. in a water bath sonicator. Doubly-distilled water was added, if necessary, to return the total volume to 1 ml. All liposomal solutions were prepared at a final concentration of 5 mg/ml. The pH of the liposomal suspension was checked by pH Boy (Camlab Ltd., Over, Cambridgeshire, UK) and adjusted to pH 7.0±0.1 with concentrated aqueous solutions of HCl and NaOH.

Liposomes were extruded (Extruder, Northern Upids, Inc., Vancouver, BC, Canada) by passing through two 100 nm polycarbonate filters (Isopore™ Membrane Filters, Millipore (UK) Ltd., Watford, Hertfordshire, UK) ten times. For each preparation, the size distribution of liposomes was measured by photon correlation spectroscopy (PCS) (Coulter® N4 Plus Submicron Particle Sizer, Beckman Coulter, High Wycombe, Buckinghamshire, UK). For phospholipid-containing liposomes, phospholipid concentrations were checked by the Stewart assay.²³ In each case, a final lipid concentration of 4.7±0.1 mg/ml was observed. For non-phospholipid containing liposomes, lipid loss was also inevitable on extrusion. For all fast-extruding liposomal suspensions, total lipid concentration was assumed to be 4.7 mg/ml. For slower extrusions, total lipid concentrations were assumed to be 4.3 mg/ml. Very slowly extruding liposomes were extruded three times only and assumed to be 4.7 mg/ml.

Formation of LMDs (Liposome:Mu:DNA)

Plasmid DNA containing the β-galactosidase gene (pNGVL1-nt-beta-gal; 7.53 kbp) was stored as frozen aliquots at −80° C., at a concentration of 1.2 mg/ml; μ (mu) peptide (adenoviral core peptide [H₂N-Met-Arg-Arg-Ala-His-His-Arg-Arg-Arg-Arg-Ala-Ser-His-Arg-Arg-Met-Arg-Gly-Gly-CO₂H]) was synthesised as described previously,⁷ and maintained at 4° C. in aliquots of 1 mg/ml. Mu-DNA complexes were made by adding plasmid DNA to mu in 4 mM HEPES at a ratio of 1:0.6 (w/w) with fast vortexing. LMDs were prepared by complexing mu-DNA particles with cationic liposomes (as prepared above) in a ratio of 1:12 (w/w), such that all LMDs for all formulations were substantially cationic. For each preparation, the size distribution of LMDs was measured by PCS.

Cell Transfections

Panc-1 cells were maintained in RPMI/10% FCS/1% penicillin-streptomycin (Gibco™, Invitrogen Corporation, Paisley, Scotland, UK) at 37° C./5% CO₂ in a humidified atmosphere. Twenty-four hours prior to transfection, 30 000 cells per well were seeded in 48 well microtitre plates (Corning Costar, Merck Ltd., Lutterworth, Leicestershire, UK) in 500 μl medium. As a control, plasmid DNA (0.5 μg in 200 μl medium) was added to a well. As a positive control, 1.5 μl Transfast™ (Promega Corporation, Madison, Wis., USA; prepared according to the supplier's protocol) in 200 μl medium was complexed with plasmid DNA (0.5 μg) and added to a well. 5 μl (0.5 μg DNA) of LMD formulation was added to each well. All experiments were performed in quadruplicate. The plate was swirled for 30 s, and incubated for 1 h at 37° C. Medium was then removed, and 500 μl of fresh medium was added. The cells were incubated at 37° C. for a further 24 h.

COS-7 cells were maintained in DMEM/10% FCS/1% penicillin-streptomycin (Gibco™) at 37° C./5% CO₂ in a humidified atmosphere. Twenty-four hours prior to transfection, 10 000 cells per well were seeded in 48-well microtitre plates (Coming Costar) in 500 μl medium. Plasmid DNA and Transfast™ controls were prepared as above. 5 μl (0.5 μg DNA) of LMD formulation was added to each well. All experiments were performed in quadruplicate. The plate was swirled for 30 s, and incubated for 1 h at 37° C. Medium was then removed, and 500 μl of fresh medium was added. The cells were incubated at 37° C. for a further 24 h.

β-Galactosidase Assay and Total Protein Determination

Medium was aspirated from the wells and the cell layer was washed with phosphate buffered saline (PBS) (Gibco™). The cells of each well were lysed in 150 μl lysis reagent (prepared according to the supplier's protocol; β-Gal Reporter Gene Assay, Roche Diagnostics GmbH, D-68305 Mannheim, Germany) at room temperature for 30 min. After lysis, equal amounts, 50 μl and 20 μl, of each cell suspension were used for the determination of β-galactosidase activity and for the determination of total cellular protein (to normalise results), respectively.

In the β-gal assay, 100 μl of the substrate reagent was added to 50 μl of the cell suspension in a white 96-well microplate (Coming Costar). The plate was incubated for 30 min. at room temperature. Automatic initiation (enhancement of enzymatic activity produced upon addition of substrate reagent) was performed by a microplate luminometer (Anthos Lucy 1, Labtech International Ltd., Ringmer, East Sussex, UK) which injected 50 μl initiation reagent and enzymatic activity was measured over the subsequent 30 s.

The amount of cellular protein was quantified in a BCA assay (Pierce, Rockford, Ill., USA) using 20 μl of the cell lysate or bovine serum albumin as internal calibration standard and adding 200 μl of BCA reagent (according to the supplier's protocol). Following an incubation period of 30 min. at room temperature, the colorimetric measurement was performed at 570 nm by means of a microplate reader (Anthos Lucy 1). β-Galactosidase activity was expressed as RLU/μg of protein. Controls and helper lipids or cationic lipids (as stated in figure titles) are presented on the x-axis in the following figures.

DOPC-Analogues

In cationic liposome formulations, DOPC is generally observed to eliminate or severely attenuate transfection, cf DOPE.²⁴ Transfection results (Panc-1 cells) of LMDs of CDAN:Helper Lipid, 1:1 (mol/mol) liposomes are shown below (FIG. 12). Transfection levels are all very low. Substituting DO(4-yne)PC for DOPC results in almost complete loss of any transfection ability. However, DO(9-yne)PC and DO(14-yne)PC transfect as well, if not better than, DOPC.

Transfection results of identical LMDs with COS-7 cells are shown in FIG. 13. DOPC and Transfast transfect equally well. Again, substituting DOPC with DO(4-yne)PC results in a fall in transfection ability. DO(14-yne)PC transfects a little better than DOPC but DO(9-yne)PC transfects around twice as well as DOPC. DO(9-yne)PC has proven to transfect as well as, if not better than, DOPC across two cell lines (Panc-1 and COS-7 cells).

DOPE-Analogues

FIG. 14 shows the transfection data (COS-7 cells) of LMDs of CDAN:Helper Lipid, 1:1 liposomes. When the helper lipid is either DOPE or DO(9-yne)PE, transfection levels are high and approximately the same; substitution with either DO(4-yne)PE or DO(14-yne)PE results in transfection falling by about 67%.

Below is the set of results obtained with Panc-1 cells (FIG. 15 ), using LMDs composed of CDAN:Helper Lipid, 3:2 liposomes. DOPE transfects around as twice as well as the nearest acetylenic analogue (DO(9-yne)PE), although absolute levels are quite low. Both DO(4-yne)PE and DO(14-yne)PE transfect very poorly. Notice the same pattern across the acetylenic analogues is observed as for FIG. 14.

The transfection results of LMDs of CDAN:Helper Lipid, 1:1 liposomes are shown below (FIG. 16) for Panc-1 cells. Within error bars, DOPE, DO(4-yne)PE and DO(9-yne)PE transfect well and approximately to the same degree. DO(14-yne)PE transfects around half as well as DOPE.

Continuing the trend in reducing the ratio of CDAN:Helper Lipid, thereby reducing the positive charges of the liposomes and LMDs, the results shown below (FIG. 17) are for LMDs of liposomes of CDAN:Helper Lipid, 2:3 molar ratio (Panc-1 cells). The transfection level of DOPE is now below that of Transfast. Interestingly, all the monoacetylenic analogues transfect better than DOPE. DO(4-yne)PE and DO(9-yne)PE transfect the best, up to five times better than DOPE. DO(14-yne)PE transfects the least well out of the monoacetylenic analogues.

FIG. 18 shows a repeat of the previous experiment (again Panc-1 cells). Liposomes were not prepared fresh but LMDs were prepared fresh. Notice there is a similar pattern, other than the fact that the transfection level of DO(4-yne)PE has fallen to around that of Transfast. Also, the relative increase in transfection on substituting DOPE with DO(9-yne)PE has lead to around a six-fold improvement. It is difficult to compare absolute values from experiment to experiment due to factors such as the stage of the cell cycle. Therefore, we included a “control” of LMDs of CDAN:DOPE, 1:1 liposomes. These LMDs transfect as well as the LMDs composed of CDAN:DO(9-yne)PE, 2:3 liposomes.

DOTAP-Analogues

FIG. 19 shows the transfection data for Panc-1 cells of LMDs composed entirely of DOTAP or DOTAP-analogue liposomes. All analogues (including standard DOTAP) transfect less well than the positive control Transfast, except for DO(14-yne)TAP. All analogues transfect better than DOTAP; DO(14-yne)TAP is a six-fold improvement on standard DOTAP.

The transfection data of LMDs of DOTAP liposomes with COS-7 cells is shown below (FIG. 20). Within the error bars, Transfast and DOTAP transfect similarly. Substituting DOTAP for DO(4-yne)TAP results in transfection falling by about 40%; substituting with DO(14-yne)TAP results in almost total loss of transfection activity. DO(9-yne)TAP transfects around one and-a-half times better than standard DOTAP. This pattern seen with the monoacetylenic analogues on going from DO(4-yne)TAP to DO(9-yne)TAP to DO(14-yne)TAP is reminiscent of that seen with the DOPE-analogues.

Below (FIG. 21) is the transfection data of LMDs of DOPE:Cationic Lipid, 1:1 liposomes (Panc-1 cells). All transfect worse than Transfast but all transfect better than DOTAP LMDs alone. Transfection levels are very low. Substituting DOTAP with DO(4 yne)TAP or DO(9-yne)TAP leads to a small increase in transfection but a seven-fold increase is observed on replacing DOTAP with DO(14-yne)TAP in DOPE:DOTAP liposomes.

Substituting DOPE with cholesterol led to the results below (FIG. 22). Low transfection levels are again observed (Panc-1 cells). Replacing DOTAP with DO(4-yne)TAP almost abolishes transfection, and with DO(14-yne)TAP results in loss of around 30% transfection activity. However, DO(9-yne)TAP transfects as well as DOTAP.

The final sets of transfection data are for LMDs whose liposomes are composed of either DOPE:Cationic Lipid, 1:1 (FIG. 23) or Cholesterol:Cationic Lipid, 1:1 (FIG. 24), COS-7 cells. Pure DOTAP liposomes (100% DOTAP) transfect better then DOTAP:Chol which transfects better than DOTAP:DOPE. All DOTAP-analogues transfect less well or to approximately the same degree when formulated as DOTAP:DOPE, 1:1.

DO(4-yne)TAP:Cholesterol, 1:1 and DO(14-yne)TAP:Cholesterol, 1:1 liposomes transfect as well as pure DOTAP liposomes. DOTAP:Cholesterol, 1:1 and DO(9-yne)TAP, 1:1 liposomes transfect only half as well as DO(4-yne)TAP:Cholesterol, 1:1 liposomes in COS-7 cells.

Summary and Conclusions

DO(9-yne)PC and DO(14-yne)PC transfect as well as DOPC in Panc-1 cells when formulated as CDAN:Helper Lipid, 1:1 (molar ratio) liposomes. DO(9-yne)PC transfects twice as well as DOPC in COS-7 cells.

DOPE and DO(9-yne)PE transfect COS-7 cells very well and to a similar degree as CDAN:Helper Lipid, 1:1 liposomes. DO(4-yne)PE and DO(14-yne)PE transfect about 1/3 as well as DOPE.

With liposomes composed of CDAN:Helper Lipid in the molar ratio 3:2, DOPE transfects better than all the monoacetylenic analogues (Panc-1 cells). The best of the analogues is DO(9-yne)PE, transfecting half as well as DOPE. All transfection levels are low, however.

Within error bars, DOPE, DO(4-yne)PE and DO(9-yne)PE all transfect equally well in CDAN:Helper Lipid, 1:1 liposomes with Panc-1 cells.

Reducing the positive charges of the LMDs further, CDAN:Helper Lipid, 2:3 liposomes transfect as well as the corresponding 1:1 liposomes, apart from when the helper lipid is DOPE. DO(4-yne)PE and DO(9-yne)PE transfect up to five times better than DOPE.

CDAN:DO(9-yne)PE, 2:3 liposomes transfect as well as CDAN:DOPE, 1:1 liposomes.

DOTAP and DOTAP:DOPE, 1:1 liposomes transfect worse than CDAN:DOPE, 1:1 liposomes. Transfection levels with DOTAP are generally poor.

Pure DOTAP-analogue liposomes transfect better than pure DOTAP liposomes. DO(14-yne)TAP liposomes transfect up to six times better than DOTAP (Panc-1 cells).

Only pure DO(9-yne)TAP liposomes transfect as well as DOTAP liposomes in COS-7 cells. DO(14-yne)TAP shows almost no transfection at all.

Pure DOTAP or DOTAP-analogue liposomes and the corresponding DOTAP:DOPE, 1:1 or DOTAP-analogue:DOPE, 1:1 liposomes exhibit a similar pattern of transfection abilities. Inclusion of DOPE in the formulation appears to impair transfection. DO(14-yne)TAP:DOPE, 1:1 liposomes transfect around seven times better than DOTAP: DOPE, 1:1 liposomes (Panc-1 cells)

With DOTAP:Cholesterol, 1:1 liposomes, DO(9-yne)TAP transfects as well as DOTAP (Panc-1 cells).

DOTAP:DOPE or DOTAP-analogue:DOPE, 1:1 liposomes all transfect less than half as well as pure DOTAP liposomes in COS-7 cells. All DOTAP:DOPE, 1:1 liposomes transfect similarly.

DOTAP:Cholesterol and DO(9-yne)TAP:Cholesterol, 1:1 liposomes transfect half as well as pure DOTAP liposomes (COS-7 cells). DO(4-yne)TAP:Cholesterol and DO(14-yne)TAP:Cholesterol, 1:1 liposomes transfect as well as pure DOTAP liposomes.

We synthesised and tested the in vitro transfection abilities of a series of monoacetylenic analogues of known oleoyl lipids. During the course of the biological evaluation of the potency of these lipids towards transfection, we have identified at least one monoacetylenic fatty acyl analogue within each series of lipid (apart from the DODAP series) that transfects as well, if not better than the corresponding, standard, oleoyl lipid. On the premise that the triple bond is more stable oxidatively than the double bond, these results suggest that those analogues which transfect as well as the standards are suitable surrogates in vitro. Moreover, the triple bond should confer greater rigidity on the liposomes, hence extend the circulation lifetimes of these liposomes in vivo. Both aspects of stability are clearly desirable.

DO(9-yne)PC (33) and DO(9-yne)PE (38) are the best phospholipid analogues, suggesting that the direct substitution of the double bond for a triple bond does not impair transfection in vitro. Moreover, not only do these lipids transfect well but it was observed that the preparation of DOPE-analogue:CDAN liposomes was easier than for the corresponding DOPE:CDAN liposomes. This may be a consequence of the opposing effects of the substantial kink in the fatty acyl chains of DOPE (encouraging fluidity) and the stiff, cholesterol-based CDAN (encouraging rigidity); on substitution of the double bond of DOPE with a triple bond, this degree of kink is greatly reduced. This may offer less resistance to the incorporation of CDAN, hence easier formulation of liposomes. Likewise, it may be more facile to include increasing amounts of DOPE-analogue, relative to CDAN.

More importantly, it has been shown that increasing the degree of neutral DOPE-analogue relative to CDAN (thereby reducing the positive charges of the cationic liposomes ad LMDs) does not impair transfection, unlike with standard DOPE. These LMDs bearing smaller positive charges offer a third degree of stability. Anionic species in the blood may be attracted electrostatically to the LMDs, potentially labelling them for destruction or, more simply, displacing the mu-DNA complex. Less charged LMDs will have longer circulation lifetimes, giving the particles the chance to reach their target tissues/cells.

Transfection data for DOTAP: DO(14-yne)TAP transfects better than DOTAP with Panc-1 cells; DO(9-yne)TAP transfects better with COS-7 cells. Different results are obtained with different helper lipids.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims

### REFERENCES

1 a. W F Anderson, Science, 1992, 256, 808.

2a. F D Ledley, Current Opinion in Biotechnology, 1994, 5, 626; K F Kozarsky, et al, ibid, 1993, 3,499; Gordon, et al., ibid, 1994, 5, 611.

3a. C P Hodgon, BioTech, 1995,13,222.

4a. P L Felgner, et al, Proc Natl Acad Sci USA, 1987, 84, 7413; Felgner, et al., Nature, 1989, 337, 387; H-J Burger, et al., Proc Natl Acad Sci USA, 1992, 89, 2145.

5a. Malone, et al., Proc Natl Acad Sci USA, 1989, 86, 6077.

6a. M-Y Chiang, et al., J Biol Chem, 1991, 226,18162.

7a. R J Debs, et al., J Biol Chem, 1990, 265,10189; C Walker, et a!, Proc Natl Acad Sci USA, 1992, 89, 7915.

8a. A D Bangham, Hospital Practice, 1992, 27, 51.

9a. J-P, Behr, et al., Proc Natl Acad Sci USA, 1989, 86, 6982; R Leventis, et a!, Biochim Biophys Acta, 1990,1023,124.

10a. X Gao, eta!, Gene Therapy, 1995, 2, 710.

11a. R Stribling, et al., Proc Natl Acad Sci USA, 1992, 89, 11277.

12a. E W F W Alton, et al., Nature Genetics, 1993, 5, 135.

References Cont'

[1] M. Ogris, S. Brunner, S. Schuller, R. Kircheis, E. Wagner, Gene Therapy 1999, 6, 595.

[2] A. L. Bailey, S. M. Sullivan, Biochimica Et Biophysica Acta-Biomembranes 2000,1468, 239.

[3] J. L. Coll, P. Chollet, E. Brambilla, D. Desplanques, J. P. Behr, M. Favrot, Hum Gene Ther 1999, 10, 1659.

[4] P. Erbacher, T. Bettinger, P. Belguise-Valladier, S. Zou, J. L. Coll, J. P. Behr, J. S. Remy, J Gene Med 1999, 1, 210.

[5]. S. M. Zou, P. Erbacher, J. S. Remy, J. P. Behr, J Gene Med 2000, 2,128.

[6] A. Bragonzi, G. Dina, A. Villa, G. Calori, A. Biffi, C. Bordignon, B. M. Assael, M. Conese, Gene Therapy 2000, 7, 1753.

[7] S. Li, M. A. Rizzo, S. Bhaftacharya, L. Huang, Gene Therapy 1998, 5, 930.

[8] M. I. Papisov, Advanced Drug Delivery Reviews 1998, 32,119.

[9] D. V. Devine, A. J. Bradley, Advanced Drug Delivery Reviews 1998, 32, 19.

[10] C. Kitson, B. Angel, D. Judd, S. Rothery, N. J. Severs, A. Dewar, L. Huang, S. C. Wadsworth, S. H. Cheng, D. M. Geddes, E. Alton, Gene Therapy 1999, 6,534.

[11] S. Li, W. C. Tseng, D. B. Stolz, S. P. Wu, S. C. Watkins, L. Huang, Gene Therapy 1999, 6, 585.

[12] N. Duzgunes, S. Nir, Advanced Drug Delivery Reviews 1999, 40,3.

[13] D. Needham, D. H. Kim, Colloids and Surfaces B-Biointerfaces 2000, 18, 183.

[14] I. M. Hafez, P. R. Cullis, Adv Drug Deliv Rev 2001, 47,139.

[15] D. Kirpotin, K. L. Hong, N. Mullah, D. Papahadjopoulos, S. Zalipsky, Febs Letters 1996, 388,115.

[16] M. N. Jones, Advanced Drug Delivery Reviews 1994, 13, 215.

[17] S. Medda, S. Mukherjee, N. Das, K. Naskar, S. B. Mahato, M. K. Basu, Biotechnology and Applied Biochemistry 1993, 17, 37.

References Cont'

(1b) Mountain, A. Trends Biotechnol. 2000 18,119-128.

(2b) Hafez, l. M. and Cullis, P. R., Adv. Drug Deliv. Rev. 2001, 47, 139-148.

(2c) Bailey, A. L. and Cullis, P. R., Biochemistry 1994, 33, 12573-12580.

(3b) Barve, J. A., Gunstone, F. D. Chem. Phys. Lipids 1971,7,311-323.

(4b) R\overline{u}rup, J. Mannova, M., Brezesinski, G., Schmid, R. D. Chem. Phys. Lipids 1994, 70,187-198.

(5b) Pisch, S., Bomscheuer, U. T., Meyer, H. H., Schmid, R. D. Tetrahedron 1997, 53,14627-14634.

(6b) Sharma, A., Sharma, U.S., Int. J. Pharm. 1997, 154,123-140.

(7b) Murray, K. D., Etheridge, C. J., Shah, S. I., Matthews, D. A., Russell, W., Gurling, H. M. D., Miller, A. D. Gene Ther. 2001, 8, 453-460.

(8b) Berry, D. E., Chan, J. A., MacKenzie, L., Hecht, S. M., Chem. Res. Toxicol. 1991, 4,195-198.

(9b) Schilstra, M. J., Nieuwenhuizen, W. F., Veldink, G. A., Vliegenthart, J. F. G., Biochemistry 1996, 35, 3396-3401.

(10b) Aitken, A. E., Roman, L. J., Loughran, P. A., de la Garza, M., Masters, B. S. S., Arch. Biochem. Biophys. 2001, 393, 329-338.

(11b) Fatope, M. O., Adoum, 0. A., Takeda, Y., Journ. Agrcul. Food Chem. 2000, 48,1872-1874.

(12b) Lenart, J., Pikula, S., Acta Biochim. Polon. 1999, 46, 203-210.

(13b) Kraus C. M., Neszmelyi A., Holly S., Wiedemann B., Nenninger A., Torssell K. B. G., Bohlin, L., Wagner, H., Journ. Nat Prod. 1998, 61, 422-427.

(14b) Datta K., Kulkami A. P., Toxicol. Lett. 1994, 73, 157-165.

(15b) Roy, B., Arruda, A., Mallik, S., Campiglia, A, 222^{nd} Amer. Chem. Soc. Nat. Meeting 26^{th}-30^{th} August 2001.

(16b) Hennies, P. T., Santana, M. H. C., Correia, C. R. D, Journ. Brazilian Chem. Soc. 2001,12,64-72.

(17b) Okada, J., Cohen, S., Langer, R. S., PCT Int. Appl. (1995), WO9503035.

(18b) Hayward, J. A., Johnston, D. S., Chapman, D., Annals New York Acad. Sciences 1985, 446, 267-281.

(19b) Patel, M., Vivien, E., Hauchecorne, M., Oudrhiri, N., Ramasawmy, R., Vigneron, J.-P., Lehn, P., Lehn, J.-M., Biochem and Biophys. Res. Commun. 2001, 281, 536-543.

References Cont'

(20b) Fieser, L. F., Fieser, M., Advanced Organic Chemistry 1961, pp. 227.

(21b) Fieser, L. F., Fieser, M., Advanced Organic Chemistry 1961, pp. 236-237.

(21c) Clayden, J., Greeves, N., Warren, S. and Wothers, P. Organic Chemistry 2001, pp.326-331.

(21d) Lewis, R. N. A. H., Mannock, D. A., McElhaney, R. N., Biochemistry 1989, 28, 541-548

(22b) Cooper, R. G., Etheridge, C. J., Stewart, L., Marshall, J., Rudginsky, S., Cheng, S. H., Miller, A. D., Chem. Eur. J. 1998, 4, 137-151.

(23b) Stewart, J. C. M., Anal. Biochem. 1959,104, 10.

(24b) Koltover, I., Salditt, T., Radler, J. O., Safinya, C. R., Science 1998, 281, 78-81.

1. A composition comprising
(i) a lipid compound comprising at least one non-polar moiety and a polar moiety,
wherein the non-polar moiety is of the formula
X-Y-Z-
wherein X is an acetylenic hydrocarbyl group containing a single C≡C bond, Y is O or CH₂, and Z is an optional hydrocarbyl group,
wherein the polar moiety is of the formula
-[T]ₘPHG,
wherein [T]ₘ is an optional group selected from C(O), NH, NR₁, NHC(O), C(O)NH,
NR₁C(O), C(O)NR, and CH₂, where R₁ is a hydrocarbyl group,
wherein PHG is a polar head group, and wherein m is the number of non-polar moieties (ii) a therapeutic agent.

2. A composition according to claim 1 wherein the compound is a neutral lipid.

3. A composition according to claim 1 wherein the compound is a cationic lipid.

4. A composition according to claim 1 wherein the compound is of the formula 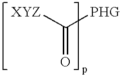
wherein p is from 1 to 10, preferably 1, 2 or 3, and wherein each X, Y and Z is selected independently of each other.

5. A composition according to claim 1 wherein the compound is of the formula

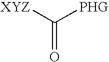

6. (original) a composition according to claim 1 comprising at least two non-polar moieties wherein each is independently selected from non-polar moieties of the formula x-y-z-.

7. A composition according to claim 2 wherein the compound is of the formula 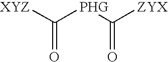
wherein each X, Y and Z is selected independently of each other.

8. A composition according to claim 6 wherein the compound is of the formula 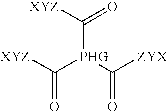
wherein each X, Y and Z is selected independently of each other.

9. A composition according to claim 1 wherein the polar head group is derived from one of phospholipids, ceramides, triacylglycerols, lysophospholipids, phosphatidylserines, glycerols, alcohols, alkoxy compounds, monoacylglycerols, gangliosides, sphingomyelins, cerebrosides, phosphatidylcholines, phosphatidylethanolamines, phosphatidylinositols (PI), diacylglycerols, Phosphatidic acids, glycerocarbohydrates, polyalcohols and phosphatidylglycerols.

10. A composition according to claim 9 wherein the polar head group is derived from a phospholipid.

11. A composition according to claim 10 wherein the phospholipid is a neutral or anionic phospholipid.

12. A composition according to claim 10 wherein the polar head group is derived from lipid selected from phosphatidylcholine (PC) phosphatidylethanolamine (PE), 3-N,N-dimethylaminopropan-1,2-diol (DAP) and 3-N,N,N-trimethylammoniopropan-1,2-diol (TAP).

13. A composition according to any one of the preceding claims claim 1 wherein the polar head group (PHG) is of the formula —W-Linker-HG, wherein W is selected from CH₂, O, NR¹ and S, wherein R¹ is H or a hydrocarbyl group, wherein Linker is an optional linker group, and HG is a head group.

14. A composition according to claim 13 wherein the head group is of the formula 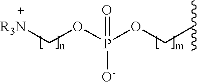
or of the formula 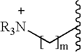
wherein R is independently selected from H and hydrocarbyl, m is from 1 to 10 and n is from 1 to 10.

15. A composition according to claim 14 wherein R is selected from H and C₁₋₆ alkyl.

16. A composition according to claim 15 wherein R is selected from H and C₁₋₃ alkyl.

17. A composition according to claim 16 wherein R is selected from H and methyl.

18. A composition according to claim 14 wherein m is from 1 to 5, preferably 1, 2 or 3.

19. A composition according to claim 14 wherein n is from 1 to 5, preferably 1, 2 or 3.

20. A composition according to claim 1 wherein X is an optionally substituted alkynyl group.

21. A composition according to claim 1 wherein X is an unsubstituted alkynyl group.

22. A composition according to claim 1 wherein X is an unsubstituted C₆-C₂₄ alkynyl group.

23. A composition according to claim 1 wherein X is an unsubstituted C₁₀-C₁₈ alkynyl group.

24. A composition according to claim 1 wherein X is an unsubstituted C₁₆ or C₁₇ alkynyl group.

25. A composition according to claim 1 wherein the C≡C of the acetylenic hydrocarbyl group is distanced from the terminal end of the acetylenic hydrocarbyl group by from 2 to 15 carbons.

26. A composition according to claim 1 wherein the C≡C of the acetylenic hydrocarbyl group is distanced from the terminal end of the acetylenic hydrocarbyl group by 2, 3, 7 or 13 carbons.

27. A composition according to claim 1 wherein Y is CH₂.

28. A composition according to claim 1 wherein when Y is CH₂, the chain X-Y-Z contains an even number of atoms;

29. A composition according to claim 1 wherein the chain X-Y-Z contains an even number of atoms.

30. A composition according to claim 1 wherein Z is an alkyl group.

31. A composition according to claim 1 wherein Z is a C₁-C₁₀, preferably C₁-C₆, preferably C₁-C₃ alkyl group.

32. A composition according to claim 1 wherein Z is —CH₂—.

33. A composition according to claim 1 wherein the compound is of the formula 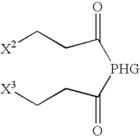
wherein X² and X³ are independently selected from unsubstituted C₁₀-C₁₈ alkynyl.

34. A composition according to claim 1 wherein the compound is of the formula 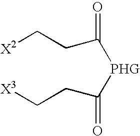
wherein X² and X³ are independently selected from unsubstituted C₁₄ alkynyl and unsubstituted C₁₅ alkynyl.

35. A composition according to claim 1 wherein the compound is of the formula 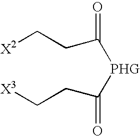
wherein X² and X³ are independently selected from CH₃(CH₂)₁₂C≡C—, CH₃CH₂CH₂C≡C(CH₂)₁₀—, CH₃(CH₂)₇C≡C(CH₂)₅—, CH₃(CH₂)₆C≡C(CH₂)₅—, and CH₃CH₂C≡C(CH₂)₁₀—.

36. A composition according to claim 33, wherein the polar head group is derived from the polar head group of a phospholipid.

37. A composition according to claim 33, wherein the polar head group is derived from the polar head group of lipid selected from phosphatidylcholine (PC) phosphatidylethanolamine (PE), 3-N,N-dimethylaminopropan-1,2-diol (DAP) and 3-N,N,N-trimethylammoniopropan-1,2-diol (TAP).

38. A composition according to claim 1 wherein the therapeutic agent is a nucleotide sequence.

39. A liposome comprising a lipid compound, wherein the lipid compound comprises at least one non-polar moiety and a polar moiety,
wherein the non-polar moiety is of the formula
X-Y-Z-
wherein X is an acetylenic hydrocarbyl group containing a single C≡C bond, Y is O or CH₂, and Z is an optional hydrocarbyl group,
wherein the polar moiety is of the formula
-[T]ₘPHG
wherein [T]ₘ is an optional group selected from C(O), NH, NR₁, NHC(O), C(O)NH, NR₁C(O), C(O)NR₁ and CH₂, where R₁ is a hydrocarbyl group,
wherein PHG is a polar head group, and wherein m is the number of non-polar moieties;
wherein the compound is other than DO(4-yne)PC, DO(9-yne)PC and DO(14-yne)PC.

40. (Cancel)

41. (Original) A lipid compound comprising at least one non-polar moiety and a polar moiety,
wherein the non-polar moiety is of the formula
X-Y-Z-
wherein X is an acetylenic hydrocarbyl group containing a single C≡C bond, Y is O or CH₂, and Z is an optional hydrocarbyl group,
wherein the polar moiety is of the formula
-[T]ₘPHG,
wherein [T]ₘ is an optional group selected from C(O), NH, NR₁, NHC(O), C(O)NH, NR₁C(O), C(O)NR₁ and CH₂, where R₁ is a hydrocarbyl group,
wherein PHG is a polar head group, and wherein m is the number of non-polar moieties;
wherein the compound is other than DO(4-yne)PC, DO(9-yne)PC, DO(14-yne)PC, DO(4-yne)PE and DO(14-yne)PE.

42. A lipid compound according to claim 41 characterised by the features of claim 2.

43. A liposome according to claim 39 in admixture with or associated with a nucleotide sequence.

44. A method of comprising administering the composition of claim 1.

45. (Cancel)

46. A method of manufacturing a medicament for the treatment of genetic disorder or condition or disease comprising using a lipid compound, wherein the compound is a lipid compound comprising at least one non-polar moiety and a polar moiety,
wherein the non-polar moiety is of the formula
X-Y-Z-
wherein X is an acetylenic hydrocarbyl group containing a single C≡C bond, Y is O or CH₂, and Z is an optional hydrocarbyl group,
wherein the polar moiety is of the formula
-[T]ₘPHG,
wherein [T]ₘ is an optional group selected from C(O), NH, NR₁, NHC(O), C(O)NH, NR₁C(O), C(O)NR₁ and CH₂, where R₁ is a hydrocarbyl group,
wherein PHG is a polar head group, and wherein m is the number of non-polar moieties.

47. A method of preparing a cationic liposome comprising forming the cationic liposome from a lipid compound comprising at least one non-polar moiety and a polar moiety,
wherein the non-polar moiety is of the formula
X-Y-Z-
wherein X is an acetylenic hydrocarbyl group containing a single C≡C bond, Y is O or CH₂, and Z is an optional hydrocarbyl group,
wherein the polar moiety is of the formula
-[T]ₘPHG,
wherein [T]ₘ is an optional group selected from C(O), NH, NR₁, NHC(O), C(O)NH, NR₁C(O), C(O)NR₁ and CH₂, where R₁ is a hydrocarbyl group,
wherein PHG is a polar head group, and wherein m is the number of non-polar moieties.

48. A pharmaceutical composition comprising a composition according to claim 1 and a pharmaceutically acceptable diluent, carrier or excipient.

49. (Cancel)

50. (Cancel)

51. (Cancel)
